(19) United States
(12) Patent Application Publication
(10) Pub. No.: US 2009/0137895 A1
(43) Pub. Date: May 28, 2009

(54) REAL-TIME DIAGNOSTIC SYSTEM EMPLOYING NON-INVASIVE METHOD TO ANALYZE ELECTRO-MAGNETIC FIELD RADIATED FROM A SUBJECT AND THE VARIATION THEREOF

(76) Inventor: Sang-Moon LEE, Seoul (KR)

Correspondence Address: JHK LAW, P.O. BOX 1078, LA CANADA, CA, 91012-1078, US

(21) Appl. No.: 12/280,766

(22) PCT Filed: Mar. 2, 2007
(86) PCT No.: PCT/KR2007/001041

§ 371 Date: Sep. 11, 2008

(30) Foreign Application Priority Data
Mar. 2, 2006 (KR) 10-2006-0020099

(51) Int. Cl. A61B 5/05

(52) U.S. Cl. 600/407

(57) ABSTRACT

The present invention relates to a real-time disease diagnostic system employing a non-invasive method capable of analyzing of an electromagnetic field and variations thereof radiated from a subject. The disease diagnostic system detects a micro electromagnetic field and variations thereof radiated from bio-action potential of the living tissue, such as cells, tissues, organs and the like of a subject, as a capacitance and variations thereof using a single- or multi-channel biosensor and the disease diagnostic system including the biosensor. The disease diagnostic system analyzes the frequency obtained according to the capacitance and variations thereof and diagnoses proliferative diseases characteristic of an abnormal cell proliferation by diagnosing whole body using a non-invasive method in a short time of 10 min to 1 hour without any side effects. The disease diagnostic system displays the result of diagnosis in the form of a numerical value, sound or three-dimensional image in real time. The disease diagnosis system may be used without any of the doctor's orders for the subject, i.e., an empty stomach or taking medicine, and used to easily confirm the removal of cancer cell by surgery and the process of treatment. Therefore, the disease diagnosis system can be useful to diagnose various diseases at an early stage in a more accurate and safe manner.

### TECHNICAL FIELD

The present invention relates to a real-time disease diagnostic system by analyzing of an electromagnetic field radiated from a subject and variation thereof, and more particularly to a real-time disease diagnostic system employing a non-invasive method capable of analyzing a micro electro-magnetic field and variation thereof radiated from cells, tissues and organs of a subject (i.e., a living organism: hereinafter, referred to as a subject). By using a material for the detection of biological electromagnetic signals (hereinafter, referred to as a biosensor), the diagnostic system is able to diagnose a health condition of a subject suffering from various diseases caused by immunodeficiency including cancer which is one of proliferative diseases characteristic of an abnormal cell proliferation at an early stage in a more accurate and safe manner by indicating the state of the diseases as a numerical value, sounds or a three-dimensional image in real time without any of side effects.

### BACKGROUND ART

Generally, there have been developed the devices for diagnosing the diverse diseases caused by immunodeficiency including cancer which is one of proliferative diseases characteristic of an abnormal cell proliferation, but the devices have disadvantages in that the devices are so bulky that they can be spatially restricted, economical burdens to the subject needed to be diagnosed, an accuracy rate of diagnosing cancer is low, i.e. from about 30% to 50%, and especially, the accuracy rate of the measured cancer is far lower than the above range if a size of the cancer is less than 1 cm, and therefore it is so difficult to recognize the presence of cancer in an early stage.

Further, the conventional diagnostic devices require the complicated doctor's orders for a subject (i.e., a living organism) in most cases before the diagnosis, i.e., an empty stomach or taking certain medicine or the like, and therefore a problem is that some diagnostic devices may cause side effects in an invasive method such as radiation penetration.

### DISCLOSURE OF THE INVENTION

### Technical Problem

Accordingly, the present invention has been made to solve the above-mentioned problems in the prior art. The diagnosis system of the present invention is made of a material for the detection of biological electromagnetic signals using specially processed bio-friendly biomaterials and the system may be medically examined by a doctor, or may be self-examined by a subject oneself according to the diagnostic site. The system also may be used to conveniently diagnose the diseases without special doctor's orders prior to the diagnosis, operated at a low power, and used to determine the diseases outside the body using a non-invasive method. Therefore, an object of the present invention is to provide a diagnostic system capable of greatly decreasing any of side effects and dramatically increasing an accuracy in the early cancer diagnosis.

Another object of the present invention is to provide a diagnostic system capable of diagnosing various diseases caused by immunodeficiency as well as early cancer, and the data base of the patients obtained through a biosensor and a disease diagnostic system including the biosensor can make hospital staffs and a subject check the long-term progress of the disease in the patients and check the result of treatment in real time, if the patient is necessarily treated for a long time.

Yet another object of the present invention is to provide a diagnostic system, unlike measurement devices such as a magnetic resonance imaging (MRI), X-Ray and computerized tomography (CT) using conventional magnetic resonance, being able to easily diagnose the presence and the kind of disease by analyzing the variation on electro-magnetic field radiated from an in vivo bio-action potential of the living organism, and the information obtained by biosensor and the system comprising the biosensor. The in vivo bio-action potential inputted in the biosensor and the system comprising the biosensor is processed in an analog circuit, converted to digital signal, numerized to allow a user to analyze the converted signal through a suitable algorism and transmitted to a display panel or monitor of the device itself or to a personal computer (PC). Therefore, it is able to analyze a health condition of the corresponding examined site of the subject, especially the presence of cancerous diseases in a prompt and accurate manner, as well as diagnosing a health condition of the subject anytime and anywhere in a prompt and simple manner without any of advance restrictions for examination or side effects. The diverse cancerous diseases may be diagnosed at an early stage, and simultaneously the results of examination may be stored in a database so that the development of the disease and the treatment result can be re-confirmed at any time. Therefore, diverse diseases such as cancer or inflammation could be diagnosed at an early stage and effectively treated and prevented the diverse diseases.

### Technical Solution

In accordance with an aspect of the present invention, there is provided a real-time disease diagnostic system capable of analyzing an electromagnetic field radiated from a subject and variation thereof, the system including a sensor probe including at least one biosensor receiving a biological electro-magnetic field in a living organism including a human body and changing a capacitance; an analog circuit for processing a bio-action potential signal measured by the sensor probe into an analog signal; a digital conversion circuit for converting the analog signal outputted from the analog circuit and processing the converted analog signal; and a power circuit for supplying driving power to the system and charging a battery with power, a communication circuit for communicating with a PC, and a communication module for wirelessly communicating with the PC.

In accordance with an aspect of the present invention, the analog circuit includes a multi-channel multiplexer for selecting a channel from the sensor probe; a sensor selection unit for selecting certain sensors among multi-channel sensors from the sensor probe and selecting a sensor for measurement from the certain sensors; a frequency controller for minimizing errors such as an error caused when a region of the biosensors of the sensor probe is composed of multiple channels, a basic manufacturing error of electronic components, and an environmental error of a measurement region and the like and adjusting a frequency generated in a frequency oscillation circuit to a reference frequency of the sensor accurately; a frequency generator for generating a reference frequency unique to the sensors based on the capacitance element when the biosensors in the sensor probe is in a normal state before their diagnosis; a frequency signal amplifier for amplifying level of a frequency signal generated from the frequency generator up to a level where the frequency signal is used in the digital conversion circuit; and a frequency allocator for allocating the frequency can be measured in the digital conversion circuit.

In accordance with an aspect of the present invention, the digital conversion circuit includes a flash memory for storing measurement data and program data; a SDRAM used for a temporary memory area; a CPU for measuring the frequency and performing various operations; a PWM module comprising a switch circuit receiving a command from a user and a buzzer generating sounds according to an input frequency; a LCD for operating measured data and displaying the operated data on a graphic user interface (GUI); and a LCD inverter for controlling a brightness of the LCD.

In accordance with an aspect of the present invention, the power circuit is free from the use of power supplied from a commercial power source, the power is supplied from a battery even though an adaptor is used for connection, and the power circuit is configured to feed back a voltage of the battery to the CPU by a battery charge measurement circuit in order to check a residual capacity and charged capacity of the battery.

In accordance with an aspect of the present invention, the frequency signal amplifier converts a level of a frequency signal outputted through a RLC circuit in the frequency generator up to a level where the frequency signal can be measured in the digital conversion circuit.

In accordance with an aspect of the present invention, the basic capacitance of the biosensor is in the range from 0.5 pF to 900 pF, and more preferably from lpF to 400 pF.

In accordance with an aspect of the present invention, a frequency of an oscillating circuit is determined according to the changes in the capacitance of the biosensor.

In accordance with an aspect of the present invention, a value of the capacitance changed according to a value of the bio-action potential detected by the biosensor and inputted from a living body tissue of the subject is converted into a frequency or a voltage value for diagnosing a disease of the subject.

In accordance with an aspect of the present invention, the health condition of the subject is determined according to a variation of a delta frequency which is a difference value between the reference frequency unique to the biosensor and the measured frequency detected by the biosensor. In accordance with an aspect of the present invention, the difference value between the reference frequency and the measured frequency is determined by determining a delta frequency using a clock of the CPU 11 supplied through an oscillator 10 and a voltage-frequency (VF) converter, measuring the frequency oscillated from the sensor, and comparing the measured frequency with the reference frequency.

In accordance with an aspect of the present invention, in the case of measuring frequency oscillated from the sensor, the frequency from sensor using an A/D converter inside or outside of the CPU is passed through frequency-voltage (FV) converter to gain a voltage value. The voltage value is converted to a digital value and measured frequency compared with the reference frequency to determine the difference value. In accordance with an aspect of the present invention, the reference frequency is a reference frequency for comparing with the measured frequency detected by the biosensor when the biosensor is free from the exposure to the bio-action potential, and is adjusted to the same frequency as the frequency unique to the biosensor.

In accordance with an aspect of the present invention, the reference frequency of the sensor as the frequency unique to the biosensor has a frequency band from 0.5 Hz to 95 MHz, and more preferably from 10 Hz to 20 MHz.

In accordance with an aspect of the present invention, the reference frequency is primarily calibrated in the analog circuit and secondarily adjusted to the same frequency as the frequency unique to the biosensor in consideration of the characteristics of the biosensor sensitive to measurement environments so that it can be adjusted by the digital conversion circuit and a given program.

In accordance with an aspect of the present invention, the reference frequency that can be adjusted in an analog mode is in the range from 5 Khz to 10 Mhz.

In accordance with an aspect of the present invention, the reference frequency that can be adjusted in a digital mode is in the range from 0.1 hz to 1 Mhz.

In accordance with an aspect of the present invention, an acquisition speed of the bio-action potential inputted from the biosensor is changed by the digital mode.

In accordance with an aspect of the present invention, the health condition of the subject is classified into three types or more according to the measured value of the delta frequency, and represented by green, yellow, red color, or other diverse color indicators, respectively.

In accordance with an aspect of the present invention, the health condition of the subject is classified into three types or more according to the measured value of the delta frequency and represented by green, yellow, red color, or other diverse color indicators, respectively, and, in the case of the subject suffering from cancer, the yellow and red indicators are randomly displayed on an LCD screen and displayed on a screen of a monitor of the PC to which data measured by the measurement device is transmitted.

In accordance with an aspect of the present invention, the health condition of the subject is classified into three types or more according to the measured value of the delta frequency and represented by green, yellow, red color, or other diverse color indicators, respectively, and boundary values among green, yellow, red color and other various colors is controlled according to the purpose of use.

In accordance with an aspect of the present invention, the health condition of the subject is classified into multiple types according to the measured value of the delta frequency and represented by a sound signal or an alarm sound that has different frequencies.

In accordance with an aspect of the present invention, the disease diagnostic system is connected with the PC by means of a wireless LAN, a wireless communication module, a USB port or RS-232C, and the PC displays the data measured by the disease diagnostic system in the form of a three-dimensional graphic, multi-dimensional graphic or stereoscopic image. Especially, in the case of the multiple channels, the diagnostic result is indicated in the form of audio signal, alarm sound, a three-dimensional graphic, a multi-dimensional graphic or a stereoscopic image, however, in the case of a single channel, the diagnostic result is indicated in the form of a digital numerical value and the audio signal or alarm sound.

In accordance with an aspect of the present invention, the disease diagnostic system is connected with the PC by a wireless communication module (LAN, Bluetooth, Zigbee), a USB port or RS 232C, and the PC stores the data measured by the disease diagnostic system into the database.

### Advantageous Effects

Accordingly, the disease diagnostic system according to the present invention has advantages, as follows.

First, the disease diagnostic system of the present invention may be useful to measure the bio-action potential signal using a single- or multi-channel biosensor, and promptly and accurately diagnose a health condition of the corresponding diagnostic region of a subject, in particular the presence of the disease such as cancer which is one of proliferative diseases characteristic of an abnormal cell proliferation in a manner, on the basis of the measured data.

Second, the conventional cancer diagnostic device requires many doctor's orders for a subject before the diagnosis, however, the disease diagnostic system of the present invention requires no limitations observed before the diagnosis, and may be useful to diagnose various diseases within a short time range from 10 minutes to 1 hour employing the non-invasive method without any of the limitations and side effects such as a vomiting symptom caused by the radiation exposure and medicine injections. Also, the disease diagnostic system of the present invention may be manufactured with low power consumption and a smaller size so that the health condition of the person in need can be diagnosed anytime and anywhere in a prompt and simple manner, thereby indicating the real-time diagnostic results in the form of the numerical value, audio sounds or three-dimensional image.

Third, the disease diagnostic system of the present invention may be useful to diagnose various diseases such as cancer which is one of proliferative diseases characteristic of an abnormal proliferation of the various cells and diverse diseases caused by immunodeficiency at an early stage. In addition, the disease diagnostic system of the present invention may be useful to re-confirm the development and treatment of the diseases at any time since every diagnosis result can be stored into the database.

Although the preferred embodiment of the present invention has been described for the purpose of illustrations, it is understood that various modifications and changes are made without departing from the scope and spirit of the invention, so that the various changes and modifications within the spirit and scope of the invention will become apparent to those skilled in the art from the detailed description and the accompanying claims.

### BRIEF DESCRIPTION OF THE DRAWINGS

The foregoing and other objects, features and advantages of the present invention will become more apparent from the following detailed description when taken in conjunction with the accompanying drawings.

FIG. 1 is a block diagram illustrating a real-time disease diagnostic system employing a non-invasive method by analyzing an electromagnetic field, and variation thereof, radiated from a subject according to the present invention.

FIG. 2 is a block diagram illustrating a digital conversion circuit of the disease diagnostic system as shown in FIG. 1.

FIG. 3 is a block diagram illustrating a sensor probe and an analog circuit of the disease diagnostic system as shown in FIG. 1.

FIG. 4 is a block diagram illustrating a power circuit of the disease diagnostic system as shown in FIG. 1.

FIG. 5 is a table illustrating a measurement order by a biosensor with a single channel (one channel) or multiple channels (five channels).

FIG. 6 is a flowchart illustrating a process of measuring a frequency using a clock of the CPU according to the present invention.

FIG. 7 is a flowchart illustrating a process of measuring a frequency using an A/D converter inside the CPU according to the present invention.

FIG. 8 is an exemplary waveform view showing the frequency generated from a frequency generator of the analog circuit.

FIG. 9 is a diagram showing a clock of the CPU corresponding to an input frequency.

FIG. 10 is a block diagram illustrating a frequency oscillation circuit and its peripheral circuits.

FIG. 11 is a flowchart illustrating a process of setting a channel frequency using a frequency controller.

FIG. 12 is a graph illustrating that a subject is diagnosed to be in a very normal state using the disease diagnostic system according to the present invention.

FIG. 13 is a graph illustrating that a subject is diagnosed to be in a normal state using the disease diagnostic system according to the present invention.

FIG. 14 is a graph illustrating that a subject is diagnosed to be in an inflammatory state using the disease diagnostic system according to the present invention.

FIG. 15 is a graph illustrating that a subject is diagnosed to be in a cancerous state using the disease diagnostic system according to the present invention.

FIG. 16 is an example that a diagnostic result by the biosensor is indicated in the form of a three-dimensional graphics on a personal computer.

FIG. 17 is a flowchart illustrating a measurement method in a case of a prescan mode among measurement modes according to the present invention.

FIG. 18 is a flowchart illustrating a measurement method in the case of a precise mode among measurement modes according to the present invention.

FIGS. 19 through 24 are exemplary views showing an image of Experimental examples.

FIG. 25 is a graph illustrating the changes of body weight in nude mice transplanted human cancer cell lines.

FIG. 26 is a graph illustrating the changes of tumor volumes in nude mice transplanted human cancer cell lines.

FIG. 27 is a graph illustrating the changes of tumor volumes in nude mice transplanted human lung cancer cell lines (G2; A549).

FIG. 28 is a graph illustrating the changes of tumor volumes in nude mice transplanted human colon cancer cell lines (G3; HCT15).

FIG. 29 is a graph illustrating the changes of tumor volumes in nude mice transplanted human melanoma cancer cell lines (G4; LOX-IMVI).

FIG. 30 is a graph illustrating the changes of tumor volumes in nude mice transplanted human prostate cancer cell lines (G5; PC-3).

FIG. 31 is a graph illustrating the changes of tumor volumes in nude mice transplanted human breast cancer cell lines (G6; MDA-MB-231).

FIG. 32 is a graph illustrating tumor weight in nude mice transplanted human lung cancer cell lines (G2; A549) on the 21^{st} day of the experiment.

FIG. 33 is a graph illustrating tumor weight in nude mice transplanted human colon cancer cell lines (G3; HCT15) on the 21^{st} day of the experiment.

FIG. 34 is a graph illustrating tumor weight in nude mice transplanted human melanoma cancer cell lines (G4; LOX-IMVI) on the 21^{st} day of an experiment.

FIG. 35 is a graph illustrating tumor weight in nude mice transplanted human prostate cancer cell lines (G5; PC-3) on the 21^{st} day of an experiment.

FIG. 36 is a graph illustrating tumor weight in nude mice transplanted human breast cancer cell lines (G6; MDA-MB-231) on the 21^{st} day of an experiment.

FIG. 37 is a picture illustrating well differentiated carcinoma of the lung cancer.

FIG. 38 is a picture illustrating manifest mitotic and gangrenous carcinoma of the colon cancer.

FIG. 39 is a picture illustrating gangrenous, undifferentiated and pleomorphic carcinoma of the melanoma.

FIG. 40 is a picture illustrating insufficiently differentiated but manifest gangrenous and pleomorphic carcinoma of the prostate cancer.

FIG. 41 is a picture illustrating insufficiently differentiated but sufficiently mitotic solid carcinoma of the breast cancer.

### MAJOR REFERENCE SIGNS OF THE DRAWINGS

1: sensor probe 2: analog circuit
3: digital conversion circuit 4: power circuit
10: oscillator 11: CPU
12: flash memory 12a: ROM selector
12b, 12c: ROM 13: SDRAM
14: LCD inverter 15: LCD
16: PWM module 16a: frequency control oscillator
16b: buzzer 17: frequency input unit
18: channel selection unit
19: communication module
19a: wireless communication module
19b: USB port
19c: RS-232C
20: frequency oscillation circuit
21: low pass filter 22: multi-channel multiplexer
23: sensor selection unit
24: frequency controller
25: frequency generator
26: frequency signal amplifier
27: frequency allocator 31: adaptor
32: battery charge measurement circuit
33: battery charging circuit
34: battery 35: 3.3 volt regulator
36: 2.5 volt regulator 37: 5 volt regulator

### BEST MODE FOR CARRYING OUT THE INVENTION

Hereinafter, exemplary embodiments of the present invention will be described with reference to the accompanying drawings.

A real-time disease diagnostic system employing a non-invasive method capable of analyzing a micro electromagnetic field, and variations thereof, radiated by a bio-action potential from cells, tissues and organs of a subject senses a micro electromagnetic field and variations thereof radiated by a bio-action potential from cells, tissues and organs of a subject as a capacitance and variations thereof using a biosensor, analyses a frequency obtained according to the capacitance and variations thereof to diagnose diverse diseases caused by immunodeficiency including cancer which is one of proliferative diseases characteristic of an abnormal cell proliferation.

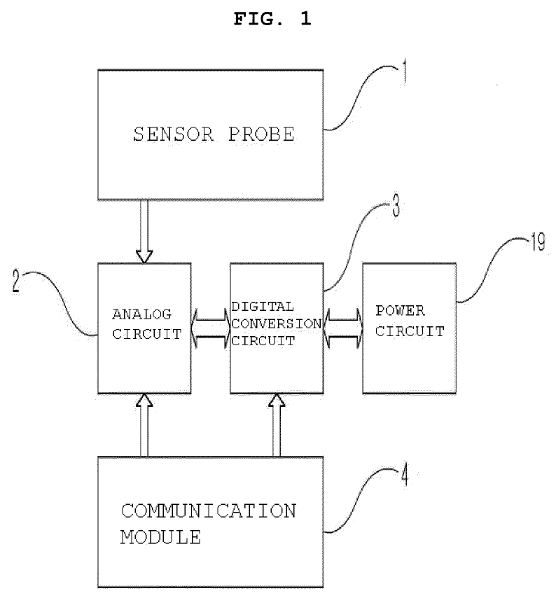

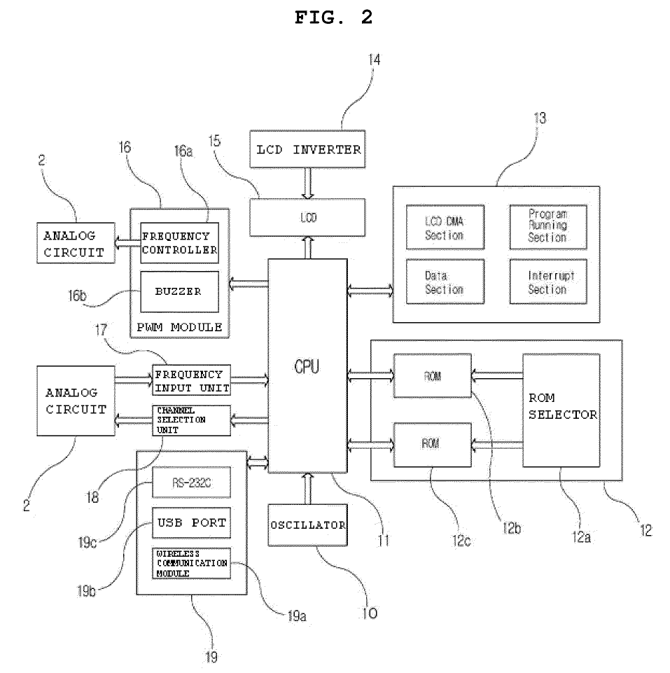

FIG. 1 is a block diagram illustrating the real-time disease diagnostic system employing the non-invasive method capable of analyzing the electro-magnetic field and variations thereof radiated from the subject according to the present invention. Here, the disease diagnostic system according to the present invention includes a sensor probe 1 comprising at least one biosensor receiving a biological electromagnetic field in a living organism including a human body and changing a capacitance; an analog circuit 2 for processing a bio-action potential signal measured by the sensor probe into an analog signal; a digital conversion circuit 3 for converting the analog signal outputted from the analog circuit 2 and processing the converted analog signal; and a power circuit 4 for supplying driving power to the system and charging a battery with power, a communication circuit for communicating with a PC, and a communication module 19 for wirelessly communicating with the PC as shown in FIG. 2.

Here, the biosensor constituting the sensor probe 1 is disclosed in the Korean Patent Application No. 10-2006-0013170 filed on Feb. 10, 2006 entitled “SENSOR FOR DETECTING BIOLOGICAL ELECTRO-MAGNETIC SIGNAL AND THE DIAGNOSTIC DEVICE USING THE SAME” filed by the same applicant of the present invention prior to filing this subject matter.

In the literature, the bio-action potential signal is generated by the bio-action potential from cells, tissues, organs and the like, of a living organism. An epidermal tissues of an animal have functions of sensing, memorizing and transmitting a extreme precision the immensely subtle minute information signal (bio-action potential signal) of the electromagnetic field. Among the epidermal tissues of an animal a scale of a fish, a scale of a reptile, a carapace of a crustacean, a cuticle of an insect species or the like is used to make an biosensor. The biosensor serves to respond to a bio-action potential signal and variations thereof and transmit and retransmit the signal and variations thereof, radiated by the bio-action potential from cells, tissues, organs and the like, of the living organism.

In addition, the bio-action potential signal sensed by the sensor probe 1 is analyzed by an algorism stored in the analog circuit 2 and the digital conversion circuit 3 and the analyzed signal is indicated in the form of three diagnostic indicators, i.e., the green or yellow indicator is displayed on a screen if there is no certain abnormal change in the bio-action potential signal, whereas the red indicator is displayed on a screen if there is a certain abnormal change in the bio-action potential signal (i.e., inflammations, etc).

Further, if the bio-action potential signal inputted by the sensor probe 1 is randomly displayed on a screen with the mixed indicators of yellow, red and other colors by the algorisms that are set in the analog circuit 2, the digital conversion circuit 3 and the CPU program in the digital conversion circuit 3, then it may be diagnosed as a cancer

Furthermore, researchers or the subject can recognize the state of the diagnosed region through an auditory sense by varying a range of the frequency applied to the buzzer 16b whenever the final result is changed into green, yellow or red colors.

After the biological electro-magnetic signal inputted into the sensor probe 1 is processed by the analog circuit 2, the digital conversion circuit 3 and so on, it can be transmitted to the PC by a wired/wireless communication module so that relevant data such as a diagnosed region, a diagnosis result and a clinical history can be classified as unique to the subject and stored in the database of the PC.

A principle of the method for diagnosing cancer at an early stage using the real-time disease diagnostic system capable of analyzing the electromagnetic field and variations thereof radiated from the subject according to the present invention is that the basic capacitance of the biosensor in the disease diagnostic system is in the range of 0.5 pF to 900 pF, and more preferably lpF to 400 pF according to the purpose of fabricating the biosensor, and the biosensor capacitance is varied according to the bio-action potential signal (electromagnetic field) inputted to the biosensor.

That is, the disease diagnostic system of the present invention may measure variation factors of the capacitance of the biosensor changed according to the bio-action potential signal, and diagnose the health condition of the subject based on the variation factors.

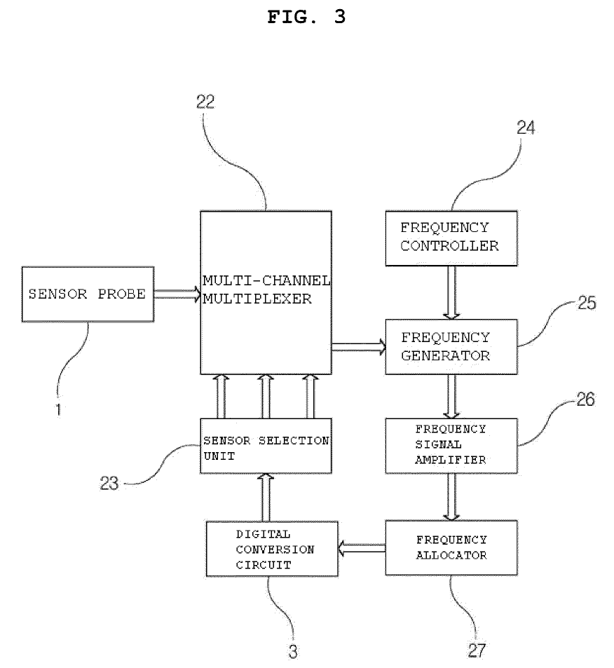

The disease diagnostic system according to the present invention includes a sensor probe 1, an analog circuit 2, a digital conversion circuit 3 and a power unit 4, as shown in FIG. 1. The above-mentioned digital conversion circuit 3 includes a CPU 11, a flash memory 12, a SDRAM 13, a frequency input unit 17, a channel selection unit 18 and the like, as shown in FIG. 2. Further, the analog circuit 2 includes a multi-channel multiplexer 22, a sensor selection unit 23, a frequency controller 24, a frequency generator 25, a frequency signal amplifier 26, a frequency allocator 27 and the like, as shown in FIG. 3.

In addition, the disease diagnostic system according to the present invention further includes a LCD 15 for displaying an operation state or diagnostic results, a LCD inverter 14, a PWM module 16, and a PC or a communication module 19 for communicating with the outside.

Hereinafter, the operations of the respective circuits will be described in more detail.

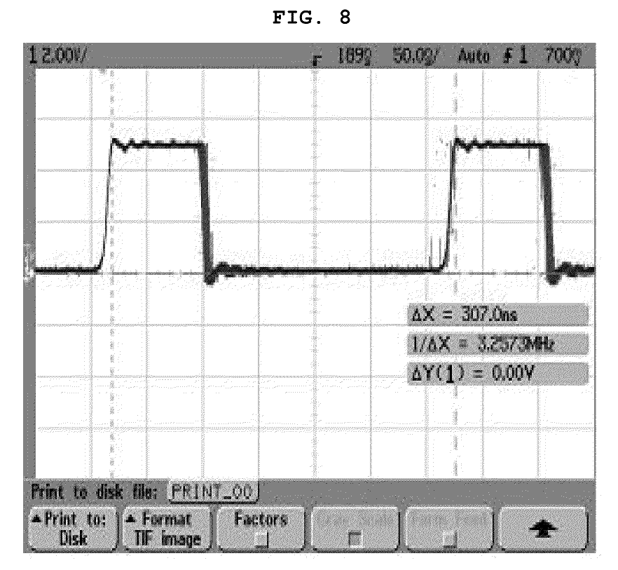

First, the frequency generator 25 of the analog circuit 2 generates a frequency as the reference frequency unique to the sensor on the basis of capacitance elements when the biosensor in the sensor probe 1 is in a normal state before the diagnosis, as shown in FIG. 8.

Here, in order for the frequency generator 25 to generate a more precise reference frequency, the reference frequency is controlled through the frequency controller 24 when the biosensor of the sensor probe 1 is in a steady state.

The biosensor of the sensor probe 1 may be fabricated with the various forms spanning from a single channel to multiple channel, as shown in FIG. 3. In the case of the multiple channels, a multi-channel multiplexer 22 for selecting a channel from the sensor probe 1 and the sensor selection unit 23 for selecting a sensor from the sensor probe 1 should be included in the system.

Since the frequency signal level generated from the frequency generator 25 is too extreme precision the immensely subtle minute signal to be inputted directly into the digital conversion circuit 3, the frequency signal amplifier 26 amplifies the extreme precision the immensely subtle minute signal up to a level that can be used in the digital conversion circuit 3.

The signal amplified by the frequency amplifier 26 is a rapid frequency on the order of several MHz range, which is then allocated by the frequency allocator 27 so that it can be measured in the digital conversion circuit 3.

The frequency signal process as above is inputted into the frequency input unit 17 and the channel selection unit 18 in the digital conversion circuit 3 shown in FIG. 2. The frequency signal inputted into the CPU 11 by the frequency input unit 17 is calculated as a frequency value by the CPU 11.

In the disease diagnostic system according to the present invention, the value of the basic capacitance of the biosensor is varied slightly during the process for manufacturing the sensor probe 1. Accordingly, although the value of the reference frequency is adjusted in the analog circuit 2 as shown in FIG. 8, the value of the basic frequency may be varied slightly due to the change in the capacitance value.

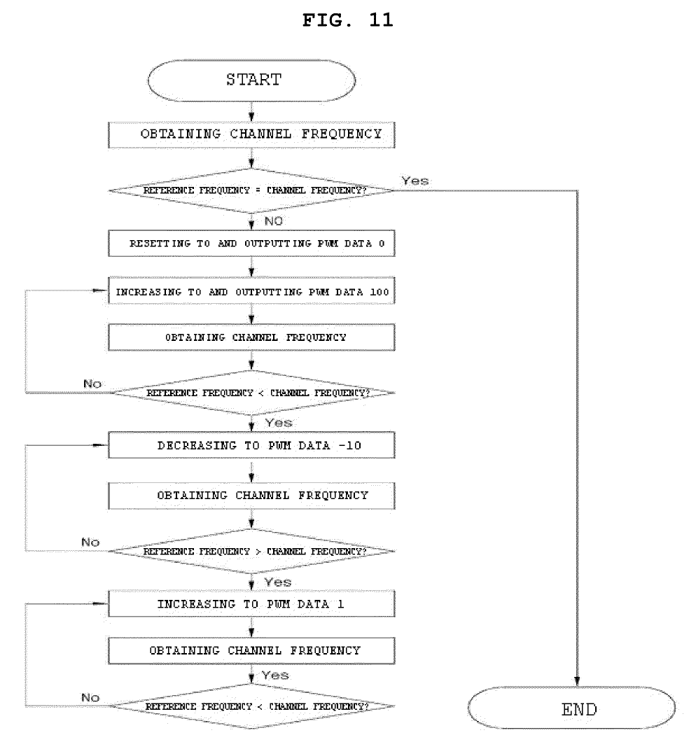

Therefore, a process of reference frequency control is carried out according to an algorithm shown in FIG. 11. That is, channels are selected by the channel frequency selection unit 18, the reference frequency of a material for the detection of biological electromagnetic signals (biosensor) for each channel is set by using the frequency control oscillator 16a, and then the CPU 11 stores sequentially voltage value of each channel to each channel in memory areas of the SDRAM 13.

That is, the change value in the PWM data is discriminated in sequence until the reference frequency coincides with the obtained channel frequency.

Then, the stored data is outputted to the digital frequency controller whenever frequencies are read from the data in each of the channel of the sensor, and the above-mentioned operation is sequentially repeated.

When the variation of the biosensor capacitance of the respective channel of the sensor probe 1 is generated (measured) owing to an input of the bio-action potential signal, the difference of the delta frequency in the reference frequency is generated, as shown in FIG. 8. The CPU 11 measures the difference between the frequencies, i.e., the value of the delta frequency, and controls the operation of the frequency controller 16a to adjust the frequency applied to the buzzer 16b in such a manner that the buzzer 16b can generate different sounds according to the value of the delta frequency.

The delta frequency means a frequency that is changed when the biosensor starts to measure the frequency (when it is in an open state, it is set to the reference frequency).

According to the present invention, there are two methods for measuring the above-mentioned delta frequency.

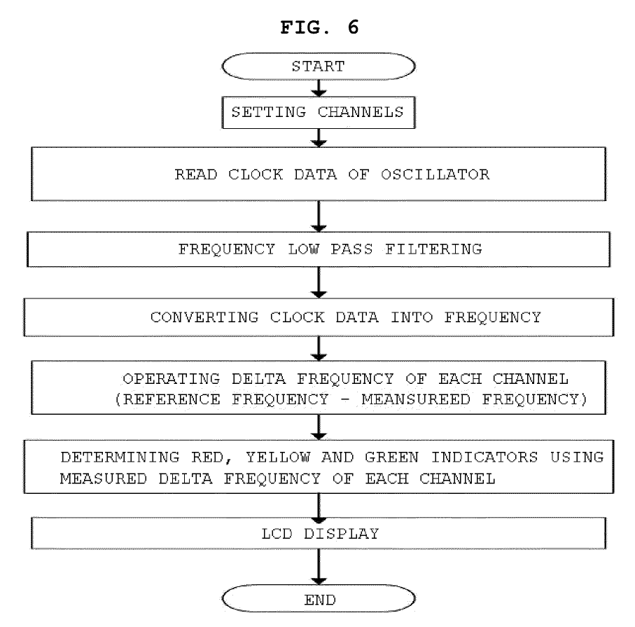

First, a method for measuring a value of the frequency by measuring difference between two vertical dotted lines shown in FIG. 8 using the clock of the CPU 11 applied through the oscillator 10, as shown in FIG. 6.

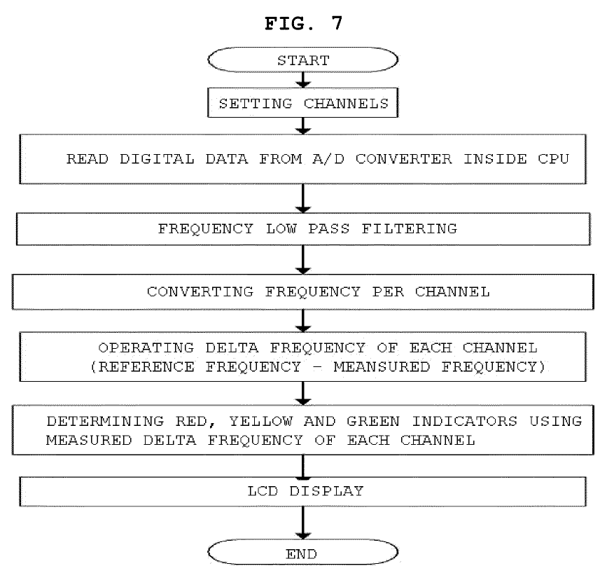

Second, a method for measuring a value of the frequency by converting a signal, generated as shown in FIG. 8 using an A/D converter (not shown) in the CPU 11, into a voltage value through a frequency-voltage (F-V) converter and converting the converted voltage value into a digital value through the A/D converter, as shown in FIG. 7.

The CPU 11 determines which color among green, yellow and red is displayed according to the value of the delta frequency. At this time, the CPU 11 allocates a region where data may be stored in a region of the flash memory 12 and stores the measured delta frequency in the allocated region. Accordingly, the LCD 15 selectively outputs green, yellow and red colors which are determined by the CPU 11 according to the value of the delta frequency.

Here, the LCD inverter 14 controls brightness of the LCD 15.

And, the CPU 11 is connected with a wireless communication module 19a and a communication module 19 including a USB port 19b and RS-232C 19c so that it can transmit the value of the delta frequency to the PC. The data transmitted to the PC using various communication schemes are displayed in the form of a three-dimensional graphics as shown in FIG. 16, or can be stored, outputted and stored into the database, by using a given program.

An operation by the analog circuit 2 to process a frequency signal will now be described.

First of all, if the bio-action potential signal of a human body or a living organism is inputted to the biosensor of the sensor probe 1, the capacitance of the biosensor is changed since the biosensor receives the biological electromagnetic field within the human body.

In order to measure variations in the capacitance, the value of the capacitance is converted into a value of the frequency by using a frequency oscillation circuit 20.

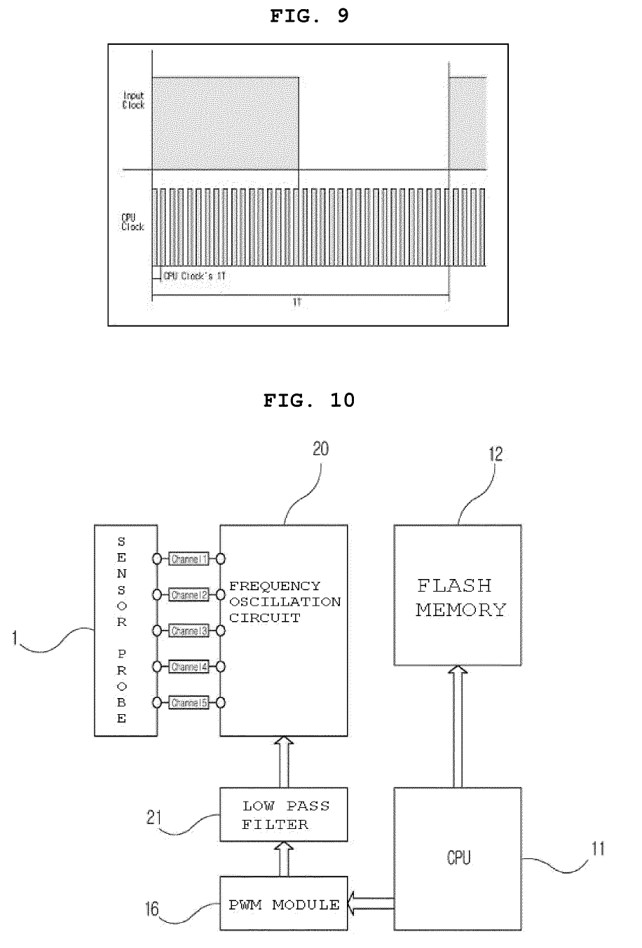

As shown in FIG. 3, the frequency controller 24 includes the frequency oscillation circuit 20, a low pass filter 21 and a PWM module 16 as shown in FIG. 10.

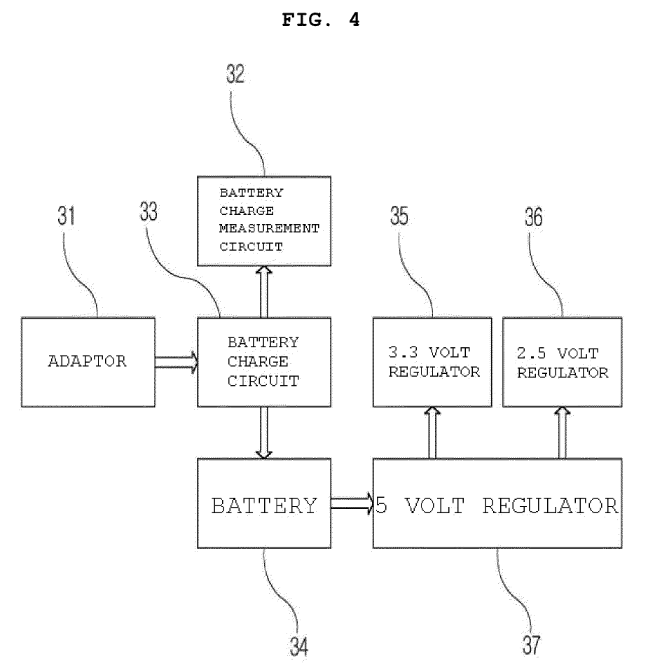

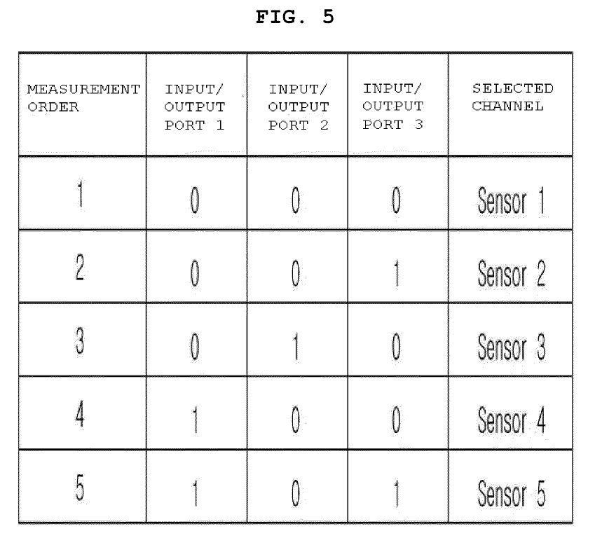

The frequency oscillation circuit 20 oscillates a reference frequency unique to the sensor of FIG. 8 when the biosensor of the sensor probe 1 is in a steady state, i.e., not in contact with the bio-action potential signal as shown in FIG. 5, the reference frequency being identical to the frequency value.

The biosensor is highly sensitive to the external environments, and therefore the frequency of the signal outputted through the frequency oscillation circuit 20 is also sensitively changed if the external environments are changed. Accordingly, the biosensor should be necessarily fixed at a uniform frequency under any of the environments when the biosensor is in an open state, i.e., when the biosensor does not measure a frequency.

Further, the frequency controller 24 is provided for minimizing errors such as an error caused when a region of the biosensors of the sensor probe 1 is composed of multiple channels, a basic manufacturing error of electronic components, and an environmental error of a measurement region and the like and adjusting a frequency generated in a frequency oscillation circuit 20 to a reference frequency of the sensor accurately.

If the bio-action potential signal of the subject, i.e. the living organism, is inputted to the biosensor of the sensor probe 1, the value of the capacitance of the biosensor is changed (increased) as the biosensor receives the biological electromagnetic field within the living organism including the human body as described above, so that the frequency is also changed. If the value of the capacitance is increased, the frequency f generated from the frequency oscillation circuit 20 is decreased according to a basic principle of a formula of

$$f = \frac{1}{2 π \sqrt{L \times C}} .$$

On the other hand, amplitude of the oscillated frequency is too extreme precision the immensely subtle minute signal so that the frequency signal amplifier 26 amplifies a signal up to a certain signal level that may be measured by the digital conversion circuit 3.

The frequency signal amplifier 26 serves to convert the frequency signal level up to a signal level of 0V to 5V and into a square wave, that may be measured in the digital converting circuit 3, since a frequency signal level outputted through the RLC circuit in the frequency generator 25 is displayed in a form of a sine wave of 1.2V to 2.2V, which is not adequate for the digital input signal used for measuring the frequency of the signal.

The biosensor of the sensor probe 1 is composed of many channels spanning from a single channel to multiple channels, and therefore a multi-channel multiplexer 22 and a sensor selection unit 23 are used to measure all of the multiple channels.

The measurement order of the biosensor that senses the bio-action potential signal using the multi-channel multiplexer 22 and the sensor selection unit 23 is as FIG. 5.

In order to allow the digital conversion circuit 3 to easily measure the signal transmitted from the analog circuit 2 to the digital conversion circuit 3, the frequency allocator 27 allocates the frequency oscillated by the frequency oscillation circuit 20.

Next, an operation of processing a frequency (signal) in the digital conversion circuit 3 will be described.

As shown in FIG. 2, the digital conversion circuit 3 includes the flash memory 12 for storing a measured data and a program data, the SDRAM 13 used for a temporary memory area, the CPU 11 for measuring a frequency and performing diverse operations, the PWM module 16 including a switch circuit for inputting a command from a user (not shown) and a buzzer 16b for generating sounds according to an input frequency, the LCD 15 for operating the measured data and displaying the operated data on a graphic user interface (GUI), and the LCD inverter 14.

The digital conversion circuit 3 needs a frequency measurement algorithm for measuring final frequencies outputted from the analog circuit 2. To measure the frequencies, the clock of the CPU 11 is counted for one period of the clock signal of the reference frequency of the material for the detection of biological electromagnetic signals (of the biosensor of FIG. 8) shown in FIG. 9, inputted through an input/output module of the CPU 11. The frequencies (F) are measured according to following Formula:

$$\begin{matrix}F = 1 / T \\ T = ( C P U Clock Counter ) \times 1 / ( C P U Frequency )\end{matrix}$$

The frequency F measured thus is divided by the frequency allocator 27 of the analog circuit 2, and then multiplied by the divided value to restore the precise value of the frequency according to a program.

When the biosensor of the sensor probe 1 is in the steady state, the frequency oscillation circuit 20 oscillates the basic frequency. However, when the biosensor of the sensor probe 1 is measuring a frequency, i.e., the bio-action potential is being inputted, the value of the capacitance is increased, and therefore the value of measurement frequency f is decreased on the basis of the formula of

$$f = \frac{1}{2 π \sqrt{L \times C}} .$$

The value of the delta frequency can be obtained by deducting the decreased frequency from the basic frequency.

Clinically speaking, the delta frequency indicates the amount of biological electromagnetic signals. Accordingly, an increase in the delta frequency indicates a large amount of biological electromagnetic signals, whereas a decrease in the delta frequency indicates a small amount of biological electromagnetic signals.

Accordingly, in the disease diagnostic system according to the present invention, the delta frequency is classified into three states. A first state where the bio-action potential is in a general motion state, a second state where the bio-action potential is in an active state, and a third state where the bio-action potential is in a very active or pulsatory state.

The respective three states are displayed as follow: “green” indicating an excellent health condition of a subject; “yellow” indicating a good health condition; and “red” indicating an inflammation or a bad health condition. That is, if the bio-action potential is in the first state, “green” is indicated on the LCD 15 of the disease diagnostic system according to the present invention or on the screen of the PC, “yellow” is indicated if the bio-action potential is in the second state, and “red” is indicated if the bio-action potential is in the third state.

When the biosensor of the sensor probe 1 is in the steady, the basic frequency generated in the frequency generator 25 by the frequency controller 24 is adjusted to the reference frequency of the frequency controller 24 shown in FIG. 3 and the biosensor shown in FIG. 8. However, There is cases, sometimes, that the reference frequency is not maintained accurately and is varied minutely in the procedure of receiving a reference frequency from the digital conversion circuit 3 and operating the received reference frequency.

The frequency control oscillator 16a is also provided in the digital conversion circuit 3 to cope with such a phenomenon as well as control the frequency precisely. As shown in FIG. 8, a frequency setting unit includes the sensor probe 1 having a material for the detection of biological electromagnetic signals, the frequency oscillation tuning circuit 20, the low pass filter 21, the CPU 11 having input and output ports, the PWM module 16, the flash memory 12 or the like.

When a power is applied to the CPU 11, the CPU 11 receives a frequency from the analog circuit 2 to confirm the reference frequency. If the measured frequency is different from the reference frequency, the CPU 11 executes an adjustment algorism on the measured frequency.

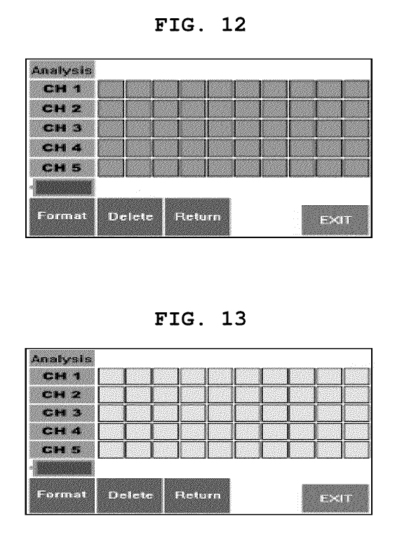

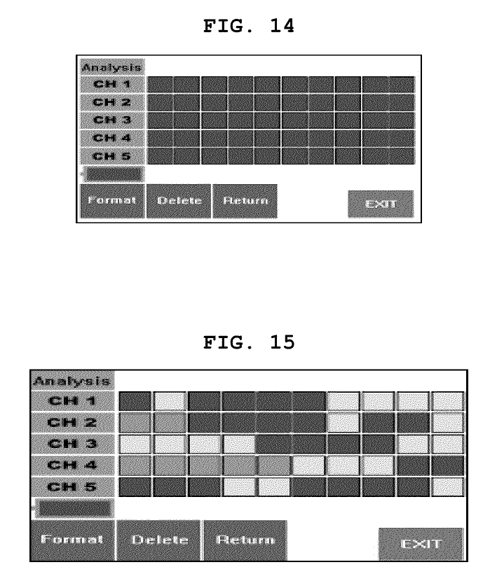

When the bio-action potential signal of the subject, i.e., the living organism is measured using the disease diagnostic system including the biosensor according to the present invention, “green” and “yellow” as shown in FIGS. 12 and 13 are maintained in a constant state if the subject is excellent or good in health, and “red” as shown in FIG. 14 is maintained in a constant state if the subject is bad in health due to an inflammation, etc.

However, in the case of cancer, the indicator states are unstable as in “red” in FIG. 14, “yellow” in FIG. 13, “red” in FIG. 14, “yellow” in FIG. 13, and “green” FIG. 12 and the indicator state is irregular as shown in FIG. 15. The degree of the irregularity, i.e., the change width of the delta frequency is different according to the individuals or the state of cancer.

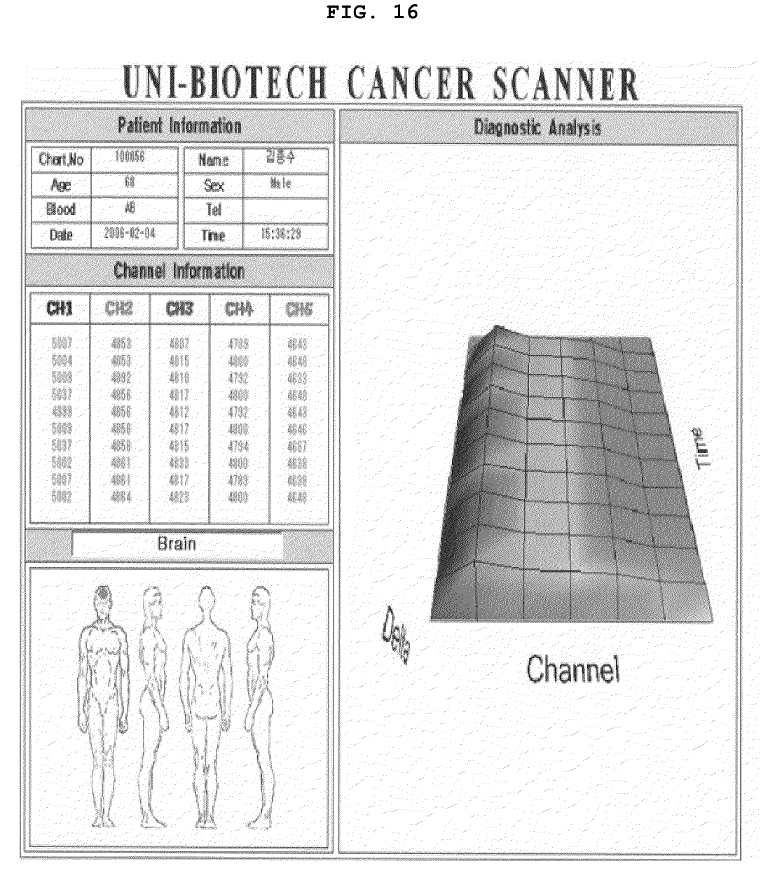

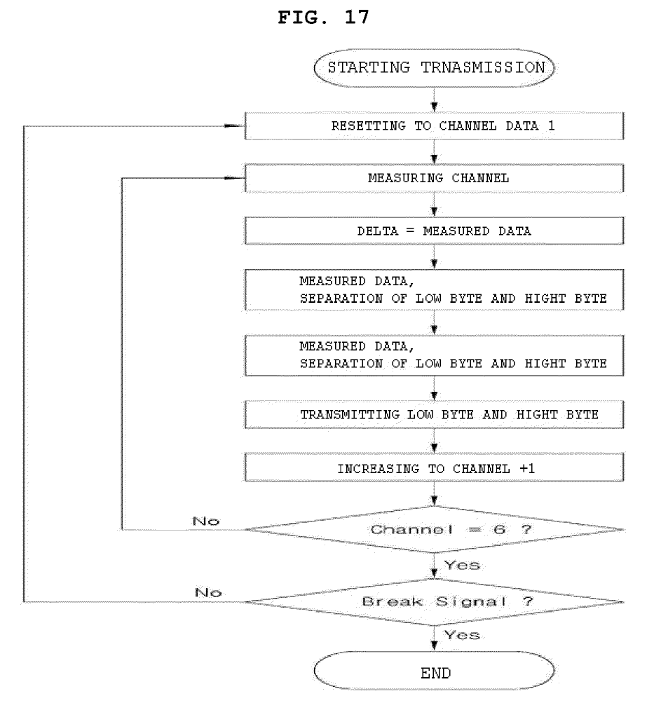

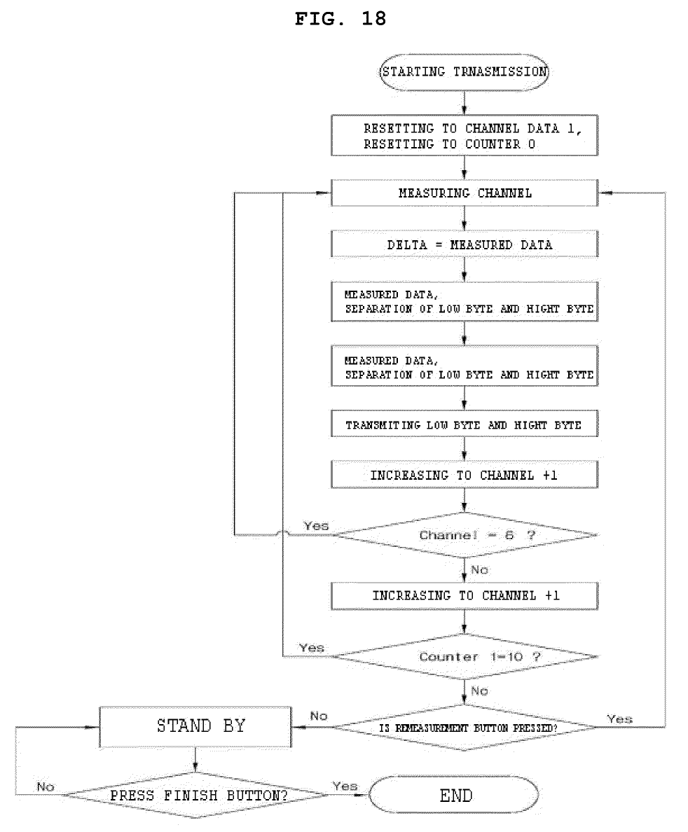

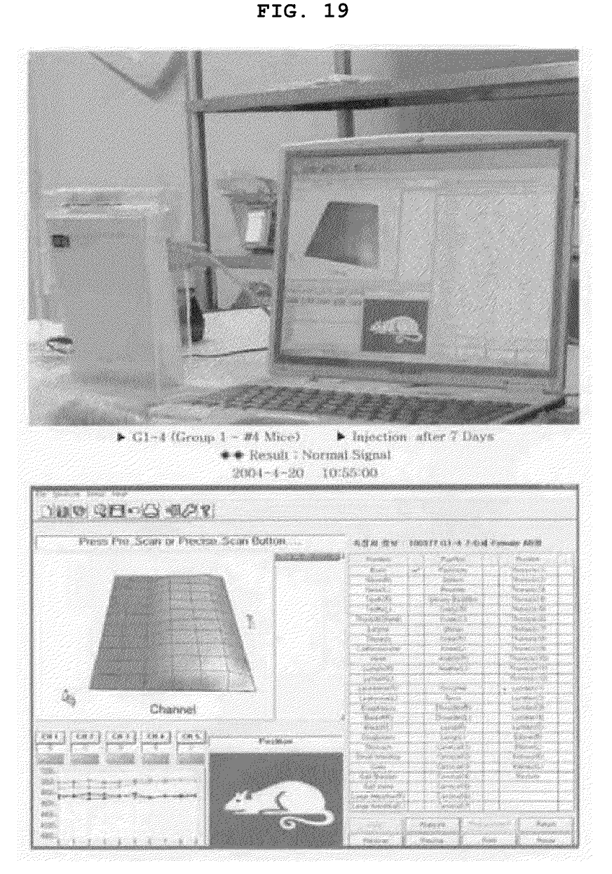

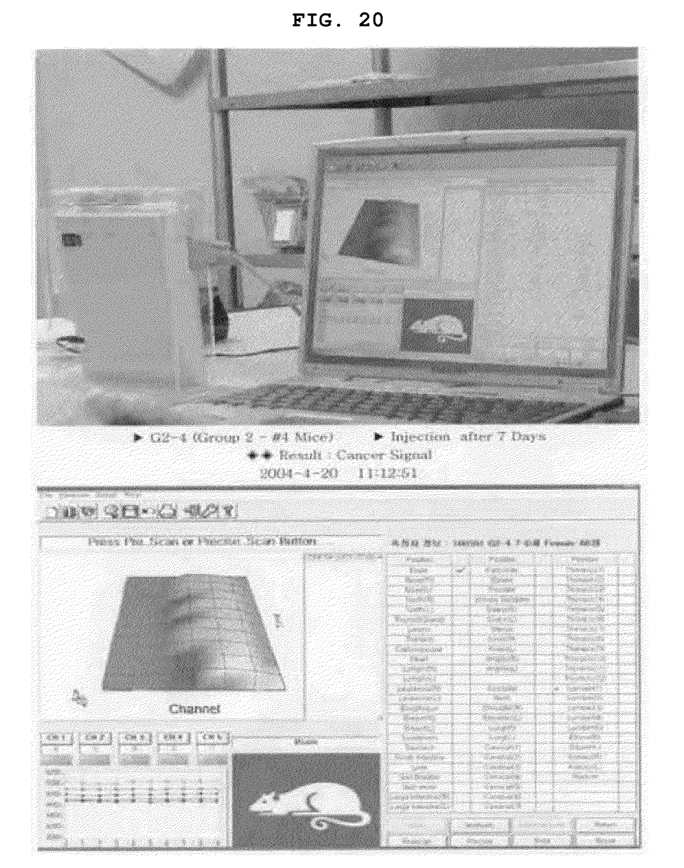

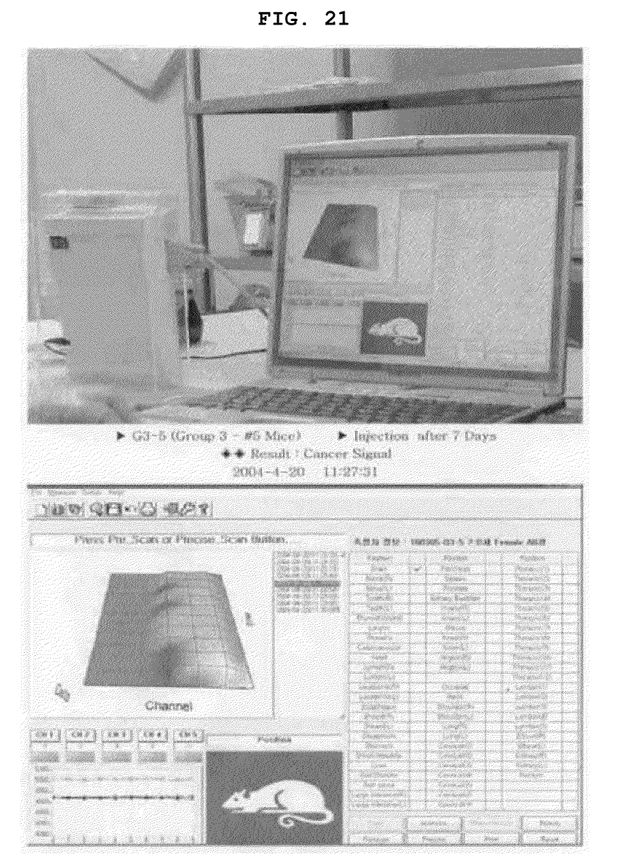

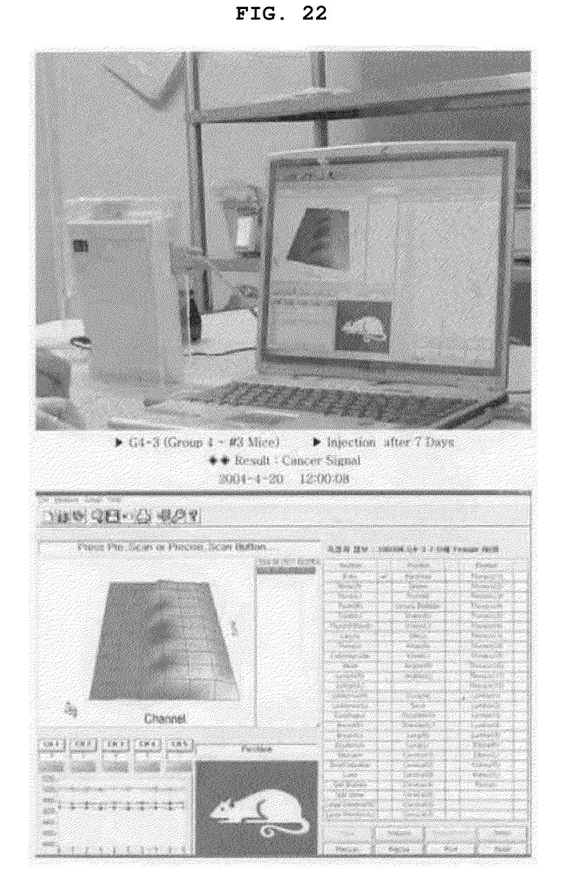

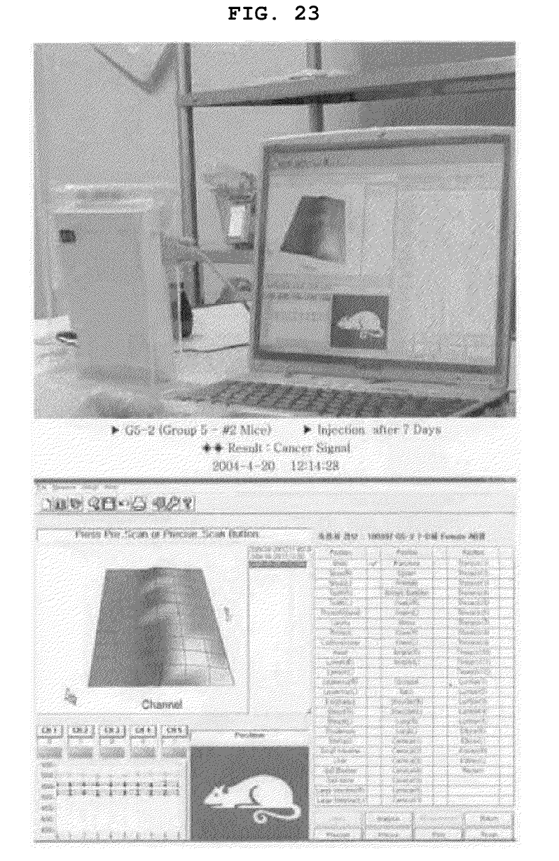

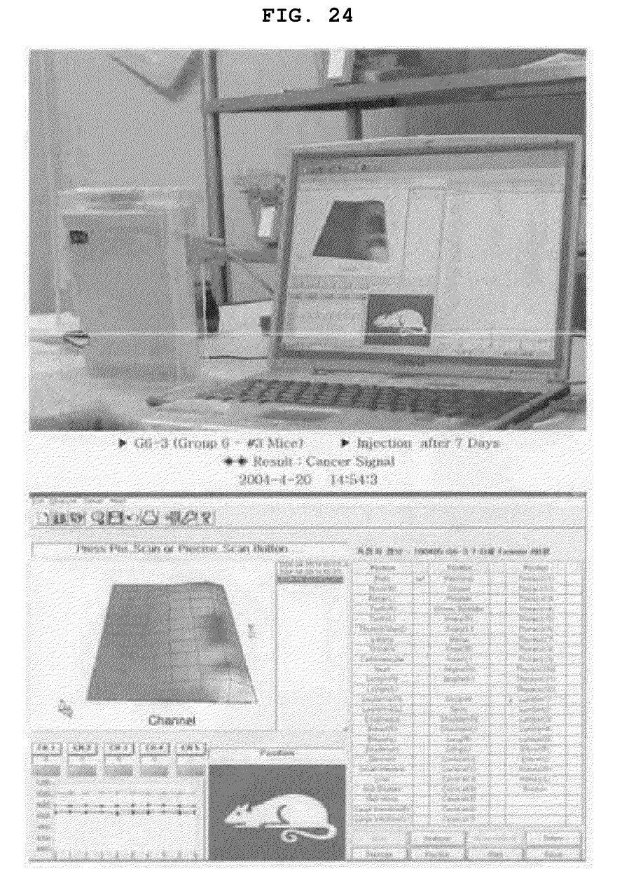

In order to measure it more precisely, frequency sampling rate is classified into three types. Specific protocols are shown in FIG. 17 and FIG. 18. FIG. 17 illustrates a channel draw mode which reads material (biosensor) frequencies at 10 ms and a prescan mode which reads material frequencies at 20 ms, and FIG. 18 illustrates a precise mode which reads material (biosensor) frequencies at 100 ms.

That is, the prescan mode as shown in FIG. 17 illustrates a process of continuously transmitting the measured data until the user presses a stop button or the PC gives a stop command when a break signal is inputted.

Meanwhile, the precision mode as shown in FIG. 18 illustrates the process of sending all data in the multi-channel biosensor ten times and then standing by until the user presses a button for measurement. The measured data as described above is displayed on the LCD 15 mounted in the PC or the disease diagnostic system. According to the delta frequency value of the measured frequency, “green (in FIG. 10)”, “yellow (in FIG. 11)” and “red (in FIG. 12)” are displayed according to the method as described above. Then, they are displayed in the form of the three-dimensional graphics on the PC, as shown in FIG. 16.

The standard of the difference value of the frequency classifying “green”, “yellow” and “red” is set on the basis of the results obtained by carrying out pre-animal clinic tests several hundreds times, which was conducted in “UNI Bio-Tec” company. As an experimental example, assume that the reference frequency is 50.40 KHz. At this time, a mouse is very good in health so “green” is indicated if the measured frequency is within the range from 50.40 to 48.38 KHz, the mouse is good in health so “yellow” is indicated if the measured frequency is within the range from 48.37 to 46.79 KHz, and the mouse is inflammatory so “red” is indicated if the measured frequency is less than 46.78 KHz. If “yellow” and “red” are irregularly indicated, it is judged to be cancer.

Upon being transmitted to the PC from the diagnostic device equipped with the material for the detection of biological electromagnetic signals (biosensor) as above, the transmitted data is transmitted according to a specific data transmission protocol by the wireless communication module 19a, the USB port 19b and the RS-232C 19c of the PC or diagnostic device.

In addition, an audio signal is outputted through the buzzer 16b so that the biological electromagnetic signal inputted from the material (biosensor) can be heard. As different sounds are generated according to colors displayed on the LCD 15, normal condition, inflammation and cancer can be judged by sounds.

The power circuit 4 of FIG. 1 is also shown in FIG. 4, and includes the adaptor 31, the battery charge measurement circuit 32, the battery charging circuit 33, the battery 34, the 3.3 volt regulator 35, the 2.5 volt regulator 36, the 5 volt regulator 37 and the like. 3.3 volt and 2.5 volt are supplied to the digital conversion circuit 3 and 5 volt is supplied to the analog circuit 2.

The battery 34 is implemented with a Nickel Metal Hybrid (Ni-MH) battery, which has a capacitance of about 1200 mA/H and thus can power the diagnostic device of the invention at a current of 550 mA for about 2 hours.

The diagnostic device of the invention is a medical device and thus does not directly use a commercial or common voltage to ensure safety for a subject (living organism). Even though the adaptor 31 is used, the diagnostic device is powered from the battery 34. To check the residual capacity and charged capacity of the battery 34, the voltage of the battery 34 is fed back to the CPU 11 by the battery charge measurement circuit 32.

### EXPERIMENTAL EXAMPLE

The measurement of cancer diagnostic performances using “functions of a biosensor of a early cancer diagnostic device and the early cancer diagnostic device” in a nude mouse transplanted with a cancer cell is conducted by Pre-Clinical Research Center (KGLP Approval) of Chemon Inc.

This experiment is implemented to evaluate a performance (effect) of a newly developed early cancer diagnostic device (a biosensor of the early cancer diagnostic device and the early cancer diagnostic device diagnostic device) for a purpose of diagnosing cancer at early stage in a nude mouse transplanted with a human cancer cell.

Specific pathogen free (SPF) Athymic BALB/C nude mice, which were 8 weeks old and females, were used as the species and cell line of a testing system in this experiment.

Test mice were grouped into six subcutaneous transplanted groups, each of which includes 10 mice, wherein the grouping being carried out by arranging the mice in order of their weight measured on the day that the mice is transplanted with the cancer cell. The mouse populations were identified using an identification label on a breeding cages and an ear punch method. However, the mouse populations were reared regardless of their groups during the experiment period, and a inventor of the present invention was not informed of the transplanted carcinoma.

Test groups are as reported in Table 1 below:

TABLE 1
<Subcutaneous Transplanted Group>

| | | | | Amount of | |
|---|---|---|---|---|---|
| | | Animal | | transplanted | |
| | | per group | Animal | cancer | |
| Group | Sex | (head) | No. | (cells/head) | Origin of cancer |
| G1 | Female | 10 | 1~10 | — | Control |
| G2 | Female | 10 | 11~20 | 0.3 × 10⁷ | Lung |
| G3 | Female | 10 | 21~30 | 0.3 × 10⁷ | Colon |
| G4 | Female | 10 | 31~40 | 0.3 × 10⁷ | Melanoma |
| G5 | Female | 10 | 41~50 | 0.3 × 10⁷ | Prostate |
| G6 | Female | 10 | 51~60 | 0.3 × 10⁷ | Breast |

* Note
G1: Control group where cancer cells are not transplanted
G2 to G6: Cancer groups where cancer cells are transplanted

This experiment is implemented to evaluate a performance of a newly developed early cancer diagnostic device (a biosensor of the early cancer diagnostic device and the early cancer diagnostic device diagnostic device) for a purpose of diagnosing cancer at early stage in a nude mouse transplanted with a human cancer cell, by using the “early cancer diagnostic device” according to the present invention.

The tests of the early cancer diagnostic device were implemented using the early cancer diagnostic device being manufactured with functions of the biosensor developed by the inventor of the present invention. In the tests, the inventor of the present invention randomly measured carcinogenesis in the animals by group from the first day of the cancer cells transplantation under the pre-clinical test regulations of the Pre-Clinical Research Center (KGLP Approval) of Chemon Inc., and Chemon measured a tumor volume from 8^{th} day after the transplantation when it is able to measure a size of the transplanted cancer cell. The functions of the biosensor and the diagnostic performances of the early cancer diagnostic device manufactured with the functions of the biosensor were compared and evaluated by comparing the experimental results measured by the inventor of the present invention and the Chemon Inc.

From the results measured by the inventor of the present invention for 7 days after the transplantation, the period of which the tumor is not observed with the naked eye, it was revealed that the early cancer diagnostic device showed a accuracy of 87.4%. From the total results obtained for the period that the tumor could be observed with the naked eye, it was revealed that the early cancer diagnostic device showed a accuracy of 95.9%.

According to the above results, the subcutaneously transplanted mouse group was tested through the total 656 experiments for three weeks using the diagnostic device manufactured with the functions of the biosensor. In the cancer diagnosis using the early cancer diagnostic device manufactured with the functions of the biosensor, hits were 166 (87.4%) out of the total 190 experiments for the first seven days after the transplantation and there was no one hit out of the total 190 experiments in which the healthy mice were diagnosed to be cancerous. According to the overall results, hits were 629 (95.9%) out of the total 656 experiments and there was no one hit out of the total 656 experiments in which the healthy mice were diagnosed to be cancerous.

### Mode for Invention

Examples of the invention will now be described in detail.

However, it should be appreciated that the following examples are to illustrate the invention but do not restrict the scope of the invention.

### Example 1

The kind of cancer cells were a human cancer cell line such as lung cancer (A549), colon cancer (HCT15), melanoma (LOX-IMVI), prostate carcinoma (PC-3), and breast cancer (MDA-MB-231), which were all obtained from the Korea Research Institute of Bioscience & Biotechnology in the form of freezing vials, and kept in a liquid nitrogen tank in a cell culture room of Pharmacology and Medicine Laboratory in the Pre-Clinical Researching center (KGLP Approval) of Chemon Inc., and each of the cancer cell lines were thawed at a 37° C. in a water bath as soon as possible. The thawed cancer cell lines were uniformly mixed in 5 ml of a RPMI1640 culture containing 10% FBS (Fetal Bovine Serum), and centrifuged at 1200 rpm for 10 minutes. 5 ml of the RPMI1640 culture was added to the separated cell pellet to prepare a cell suspension, and the cell suspension was put into a 25 cm² flask for cell culture, and then cultured in a CO₂ incubator at 37° C.

The cultured cancer cell was diluted in the physiological saline as 1×10⁷ cells/ml density and injected 0.3 ml/mice as a route of subcutaneous. The control group was injected with the same quantity and route with the saline.

The tumor volume was continuously measured using vernier calipers during a period from the 8^{th} day to the 3rd week after the subcutaneous transplantation of the cancer cell, and calculated according to following formula:

Tumor Volume (mm³)=long diameter×short diameter×height/2

The hypodermic tumor was extracted from all the mice on an autopsy day. As a result, it was seen that the tumors were induced in all of the mice, and their histopathological examination showed that the induced tumors were proven to be cancer.

When common symptoms were observed, no special symptoms were found from the animals except for those symptoms specific to cancer growth. In the group where melanoma was transplanted, mice died by one, respectively, on the 15^{th} and 18^{th} days.

### Measurement of Cancer Using Diagnostic Device

TABLE 2
<Results measured from 1^{st} day to 7^{th} day>

| | G1 | G2 | G3 | G4 | G5 | G6 | Total |
|---|---|---|---|---|---|---|---|
| Total | 30 | 35 | 35 | 30 | 30 | 30 | 190 |
| Measurement No. | | | | | | | |

Measurement Result (accuracy: 87.4%)

| | | | | | | |
|---|---|---|---|---|---|---|
| Normal (N) | 28 | 0 | 1 | 1 | 0 | 0 |
| Inflammation (I) | 2 | 7 | 2 | 3 | 3 | 5 |
| Cancer (C) | 0 | 28 | 32 | 26 | 27 | 25 |

TABLE 3
<Results measured from 1^{st} day to final day>

| | G1 | G2 | G3 | G4 | G5 | G6 | Total |
|---|---|---|---|---|---|---|---|
| Total | 115 | 115 | 115 | 101 | 105 | 105 | 656 |
| Measurement No. | | | | | | | |

Measurement Result (Accuracy: 95.9%)

| | | | | | | |
|---|---|---|---|---|---|---|
| Normal (N) | 111 | 0 | 1 | 1 | 0 | 0 |
| Inflammation (I) | 4 | 7 | 2 | 3 | 3 | 6 |
| Cancer (C) | 0 | 108 | 112 | 97 | 102 | 99 |

* Number of measurement in mice transplanted cancer cell lines at early time (from 0 day to 7 day). (N was normal, I was inflammation, and C was cancer)

| | | | Animal No. | | | | | | | | | | |
|---|---|---|---|---|---|---|---|---|---|---|---|---|---|
| | | | 1 | 2 | 3 | 4 | 5 | 6 | 7 | 8 | 9 | 10 | Total |
| G1 | No. of | | 3 | 3 | 3 | 3 | 3 | 3 | 3 | 3 | 3 | 3 | 30 |
| | Measurement | | | | | | | | | | | | |
| | Measurement | N | 3 | 3 | 3 | 3 | 3 | 3 | 3 | 2 | 3 | 2 | 28 |
| | result | I | 0 | 0 | 0 | 0 | 0 | 0 | 0 | 1 | 0 | 1 | 2 |
| | | C | 0 | 0 | 0 | 0 | 0 | 0 | 0 | 0 | 0 | 0 | 0 |
| G2 | No. of | | 4 | 4 | 4 | 4 | 4 | 3 | 3 | 3 | 3 | 3 | 35 |
| | Measurement | | | | | | | | | | | | |
| | Measurement | N | 0 | 0 | 0 | 0 | 0 | 0 | 0 | 0 | 0 | 0 | 0 |
| | result | I | 1 | 1 | 1 | 0 | 1 | 0 | 0 | 1 | 2 | 0 | 7 |
| | | C | 3 | 3 | 3 | 4 | 3 | 3 | 3 | 2 | 1 | 3 | 28 |
| G3 | No. of | | 4 | 4 | 4 | 4 | 4 | 3 | 3 | 3 | 3 | 3 | 35 |
| | Measurement | | | | | | | | | | | | |
| | Measurement | N | 0 | 0 | 0 | 0 | 0 | 0 | 0 | 0 | 0 | 1 | 0 |
| | result | I | 1 | 0 | 0 | 0 | 1 | 0 | 0 | 0 | 0 | 0 | 2 |
| | | C | 3 | 4 | 4 | 4 | 3 | 3 | 3 | 3 | 3 | 2 | 32 |
| G4 | No. of | | 3 | 3 | 3 | 3 | 3 | 3 | 3 | 3 | 3 | 3 | 30 |
| | Measurement | | | | | | | | | | | | |
| | Measurement | N | 0 | 0 | 0 | 0 | 1 | 0 | 0 | 0 | 0 | 0 | 1 |
| | result | I | 0 | 1 | 0 | 0 | 0 | 0 | 0 | 1 | 0 | 1 | 3 |
| | | C | 3 | 2 | 3 | 3 | 2 | 3 | 3 | 2 | 3 | 2 | 26 |
| G5 | No. of | | 3 | 3 | 3 | 3 | 3 | 3 | 3 | 3 | 3 | 3 | 30 |
| | Measurement | | | | | | | | | | | | |
| | Measurement | N | 0 | 0 | 0 | 0 | 0 | 0 | 0 | 0 | 0 | 0 | 0 |
| | result | I | 0 | 1 | 0 | 0 | 1 | 0 | 0 | 1 | 0 | 0 | 3 |
| | | C | 3 | 2 | 3 | 3 | 2 | 3 | 3 | 2 | 3 | 3 | 27 |
| G6 | No. of | | 3 | 3 | 3 | 3 | 3 | 3 | 3 | 3 | 3 | 3 | 30 |
| | Measurement | | | | | | | | | | | | |
| | Measurement | N | 0 | 0 | 0 | 0 | 0 | 0 | 0 | 0 | 0 | 0 | 0 |
| | result | I | 0 | 0 | 1 | 0 | 2 | 1 | 0 | 0 | 0 | 1 | 5 |
| | | C | 3 | 3 | 2 | 3 | 1 | 2 | 3 | 3 | 3 | 2 | 25 |
| G7 | No. of | | 8 | 8 | 8 | 8 | 8 | 7 | 7 | 7 | 7 | 7 | 75 |
| | Measurement | | | | | | | | | | | | |
| | Measurement | N | 7 | 7 | 8 | 8 | 8 | 5 | 7 | 6 | 4 | 5 | 65 |
| | result | I | 1 | 1 | 0 | 0 | 0 | 2 | 0 | 1 | 3 | 2 | 10 |
| | | C | 0 | 0 | 0 | 0 | 0 | 0 | 0 | 0 | 0 | 0 | 0 |
| G8 | Measurement | | 7 | 7 | 7 | 7 | 7 | 7 | 7 | 7 | 7 | 7 | 70 |
| | times | | | | | | | | | | | | |
| | Measurement | N | 0 | 0 | 0 | 0 | 0 | 0 | 0 | 0 | 0 | 0 | 0 |
| | result | I | 0 | 0 | 1 | 2 | 0 | 1 | 2 | 0 | 2 | 1 | 9 |
| | | C | 7 | 7 | 6 | 5 | 7 | 6 | 5 | 7 | 5 | 6 | 61 |

Number of measurement in mice transplanted cancer cell lines during 4 weeks. (N was normal, I was inflammation, and C was cancer)

| | | | Animal No. | | | | | | | | | | |
|---|---|---|---|---|---|---|---|---|---|---|---|---|---|
| | | | 1 | 2 | 3 | 4 | 5 | 6 | 7 | 8 | 9 | 10 | Total |
| G1 | No. of | | 12 | 12 | 12 | 12 | 12 | 11 | 11 | 11 | 11 | 11 | 115 |
| | Measurement | | | | | | | | | | | | |
| | Measurement | N | 12 | 12 | 11 | 12 | 12 | 11 | 11 | 10 | 11 | 9 | 111 |
| | result | I | 0 | 0 | 1 | 0 | 0 | 0 | 0 | 1 | 0 | 2 | 4 |
| | | C | 0 | 0 | 0 | 0 | 0 | 0 | 0 | 0 | 0 | 0 | 0 |
| G2 | No. of | | 13 | 13 | 13 | 13 | 13 | 10 | 10 | 10 | 10 | 10 | 115 |
| | Measurement | | | | | | | | | | | | |
| | Measurement | N | 0 | 0 | 0 | 0 | 0 | 0 | 0 | 0 | 0 | 0 | 0 |
| | result | I | 1 | 1 | 1 | 0 | 1 | 0 | 0 | 1 | 2 | 0 | 7 |
| | | C | 12 | 12 | 12 | 13 | 12 | 10 | 10 | 9 | 8 | 10 | 108 |
| G3 | No. of | | 13 | 13 | 13 | 13 | 13 | 10 | 10 | 10 | 10 | 10 | 115 |
| | Measurement | | | | | | | | | | | | |
| | Measurement | N | 0 | 0 | 0 | 0 | 0 | 0 | 0 | 0 | 0 | 1 | 1 |
| | result | I | 1 | 0 | 0 | 0 | 1 | 0 | 0 | 0 | 0 | 0 | 2 |
| | | C | 12 | 13 | 13 | 13 | 12 | 10 | 10 | 10 | 10 | 9 | 112 |
| G4 | No. of | | 7 | 12 | 9 | 12 | 12 | 10 | 10 | 10 | 10 | 9 | 111 |
| | Measurement | | | | | | | | | | | | |
| | Measurement | N | 0 | 0 | 0 | 0 | 1 | 0 | 0 | 0 | 0 | 0 | 1 |
| | result | I | 0 | 1 | 0 | 0 | 0 | 0 | 0 | 1 | 0 | 1 | 3 |
| | | C | 7 | 11 | 9 | 12 | 11 | 10 | 10 | 9 | 10 | 8 | 97 |
| G5 | No. of | | 11 | 11 | 11 | 11 | 11 | 10 | 10 | 10 | 10 | 10 | 105 |
| | Measurement | | | | | | | | | | | | |
| | Measurement | N | 0 | 0 | 0 | 0 | 0 | 0 | 0 | 0 | 0 | 0 | 0 |
| | result | I | 0 | 1 | 0 | 0 | 1 | 0 | 0 | 1 | 0 | 0 | 3 |
| | | C | 11 | 10 | 11 | 11 | 10 | 10 | 10 | 9 | 10 | 10 | 102 |
| G6 | No. of | | 11 | 11 | 11 | 11 | 11 | 10 | 10 | 10 | 10 | 10 | 105 |
| | Measurement | | | | | | | | | | | | |
| | Measurement | N | 0 | 0 | 0 | 0 | 0 | 0 | 0 | 0 | 0 | 0 | 0 |
| | result | I | 0 | 0 | 1 | 1 | 2 | 1 | 0 | 0 | 0 | 1 | 6 |
| | | C | 11 | 11 | 10 | 10 | 9 | 9 | 10 | 10 | 10 | 9 | 99 |
| G7 | No. of | | 5 | 5 | 5 | 5 | 5 | 6 | 6 | 6 | 6 | 6 | 55 |
| | Measurement | | | | | | | | | | | | |
| | Measurement | N | 4 | 4 | 5 | 5 | 5 | 5 | 6 | 5 | 6 | 5 | 50 |
| | result | I | 1 | 1 | 0 | 0 | 0 | 1 | 0 | 1 | 0 | 1 | 5 |
| | | C | 0 | 0 | 0 | 0 | 0 | 0 | 0 | 0 | 0 | 0 | 0 |
| G8 | No. of | | 4 | 4 | 4 | 4 | 4 | 6 | 6 | 6 | 6 | 6 | 50 |
| | Measurement | | | | | | | | | | | | |
| | Measurement | N | 0 | 0 | 0 | 0 | 0 | 0 | 0 | 0 | 0 | 0 | 0 |
| | result | I | 0 | 0 | 1 | 2 | 0 | 0 | 1 | 0 | 1 | 1 | 6 |
| | | C | 4 | 4 | 3 | 2 | 4 | 6 | 5 | 6 | 5 | 5 | 44 |
| | | | Animal No. | | | | | | | | | | |
| | | | 11 | 12 | 13 | 14 | 15 | 16 | 17 | 18 | 19 | 20 | Total |
| G7 | No. of | | 7 | 7 | 7 | 7 | 7 | 12 | 12 | 12 | 12 | 12 | 95 |
| | Measurement | | | | | | | | | | | | |
| | Measurement | N | 7 | 7 | 7 | 7 | 7 | 7 | 10 | 11 | 5 | 9 | 67 |
| | result | I | 0 | 0 | 0 | 0 | 0 | 5 | 2 | 1 | 7 | 3 | 18 |
| | | C | 0 | 0 | 0 | 0 | 0 | 0 | 0 | 0 | 0 | 0 | 0 |
| G8 | No. of | | 7 | 7 | 7 | 7 | 7 | 12 | 12 | 12 | 12 | 12 | 95 |
| | Measurement | | | | | | | | | | | | |
| | Measurement | N | 0 | 0 | 0 | 0 | 0 | 0 | 0 | 0 | 0 | 0 | 0 |
| | result | I | 0 | 0 | 0 | 0 | 1 | 1 | 1 | 0 | 1 | 0 | 4 |
| | | C | 7 | 7 | 7 | 7 | 6 | 11 | 11 | 12 | 11 | 12 | 91 |

(1) A control group wasn't injected with any cancer cells. They turned to have no natural occurring cancer until the end of the experiment. Therefore, we only counted the case as a correct when the cancer detector diagnosed the control mice as normal. The rest that is stress or inflammation or cancer were counted as incorrect.

(2) The cancer-transplanted group has been proved to have the cancer 100% by histopathological test. Therefore, we only counted the case as correct when cancer detector diagnosed as the cancer and the rest that is stress or inflammation or normal has been counted as incorrect.

(3) Up until 7 days after transplantation, the tumor was invisible to the naked eyes (a total of 190 times) with an accuracy of 87.4% and the official (a total of 656 times) accuracy is 95.9%. Moreover, cancer was not detected in any of the mice in the control group.

After the cancer cell was transplanted into the nude mouse, the initial diagnosis and the presence of cancer were measured using the early cancer diagnostic device manufactured with the functions of the biosensor. The inventor of the present invention measured carcinogenesis in the animals by group from the first day of the cancer cell transplantation under the pre-clinical test regulations of the Pre-Clinical Research Center (KGLP Approval) of Chemon Inc. by using the early cancer diagnostic device manufactured with the functions of the biosensor. And, the Chemon measured a tumor volume from 8^{th} day after the transplantation when it is able to measure a size of the transplanted cancer cell. The experimental results measured by the inventor of the present invention and the Chemon Inc. were compared with each other to calculate a diagnostic accuracy of the early cancer diagnostic device manufactured with the functions of the biosensor.

The tumor was induced in all of the subcutaneously transplanted animals (100%), and was proven to be pathologically cancer. The accuracy calculated using the early cancer diagnostic device manufactured with the functions of the biosensor was 95.99%, and there was no one experiment in which the control group (healthy mice) was diagnosed to be cancer.

According to the results as described above, in the experiment of measuring an ability to diagnose a cancer using the early cancer diagnostic device manufactured with the functions of the biosensor, hits were 166 out of the total 190 experiments for the first seven days after the transplantation, which showed a accuracy of 87.4%. According to the overall results, hits were 629 out of the total 656 experiments, which showed a accuracy of 95.9%. And, there was no one hit out of the total 656 experiments in which the healthy mice were diagnosed to be cancer.

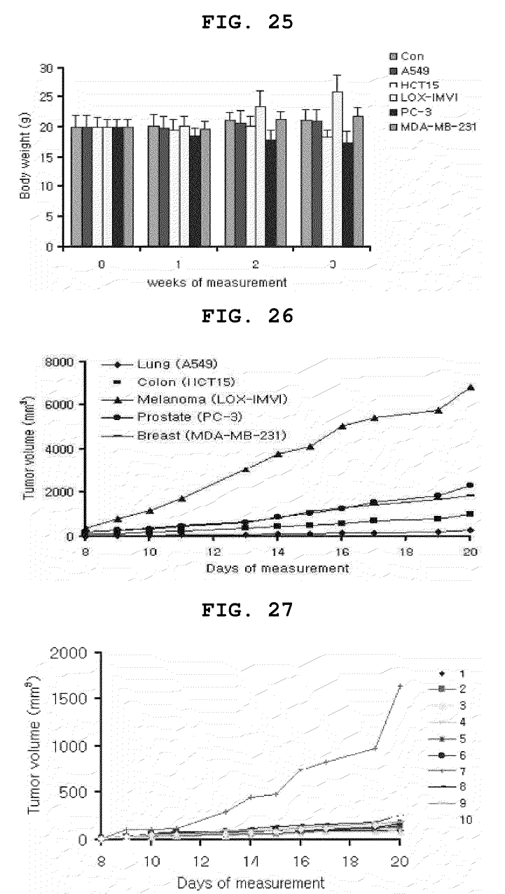

FIG. 25 illustrates the changes of body weight in nude mice transplanted human cancer cell lines. Here, the cancer cells were transplanted using a hypodermic transplantation method, and the change in body weight were measured at the same time when a mouse was transplanted with the cancer cells.

FIG. 26 illustrates the changes of tumor volumes in nude mice transplanted human cancer cell lines. Here, a tumor size was measured from the 8^{th} day after the transplantation of the human cancer cell.

FIG. 27 illustrates the changes of tumor volumes in nude mice transplanted human lung cancer cell lines (G2; A549). Here, a tumor size was measured from the 8^{th} day after the transplantation of the cancer cell, the cancer cell was diluted in saline to a density of 1×10⁷ cells/ml, and an amount of the administered solution was 0.3 ml. The numbers as shown in the right legend on the graph are Nos. of the measured mice.

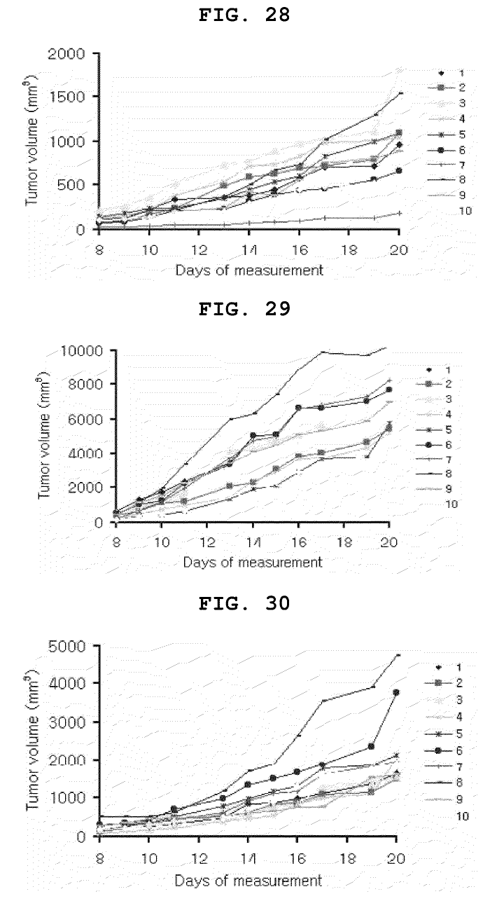

FIG. 28 illustrates the changes of tumor volumes in nude mice transplanted human colon cancer cell lines (G3; HCT15). Here, a tumor size was measured from the 8^{th} day after the transplantation of the cancer cell, the cancer cell was diluted in saline to a density of 1×10⁷ cells/ml, and an amount of the administered solution was 0.3 ml. The numbers as shown in the right legend on the graph are Nos. of the measured mice.

FIG. 29 illustrates the changes of tumor volumes in nude mice transplanted human melanoma cancer cell lines (G4; LOX-IMVI). Here, a tumor size was measured from the 8^{th} day after the transplantation of the cancer cell, the cancer cell was diluted in saline to a density of 1×10⁷ cells/ml, and an amount of the administered solution was 0.3 ml. The numbers as shown in the right legend on the graph are Nos. of the measured mice.

FIG. 30 illustrates the changes of tumor volumes in nude mice transplanted human prostate cancer cell lines (G5; PC-3). Here, a tumor size was measured from the 8^{th} day after the transplantation of the cancer cell, the cancer cell was diluted in saline to a density of 1×10⁷ cells/ml, and an amount of the administered solution was 0.3 ml. The numbers as shown in the right legend on the graph are Nos. of the measured mice.

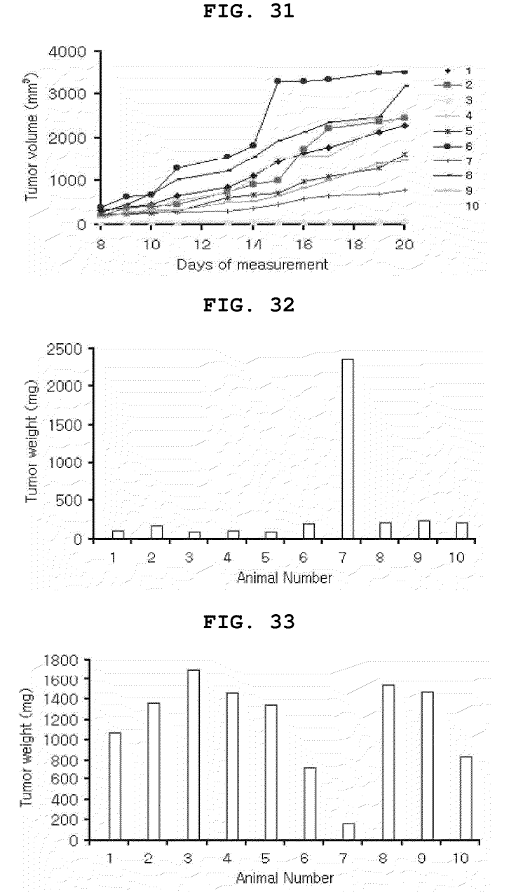

FIG. 31 illustrates the changes of tumor volumes in nude mice transplanted human breast cancer cell lines (G6; MDA-MB-231). Here, a tumor size was measured from the 8^{th} day after the transplantation of the cancer cell, the cancer cell was diluted in saline to a density of 1×10⁷ cells/ml, and an amount of the administered solution was 0.3 ml. The numbers as shown in the right legend on the graph are Nos. of the measured mice.

FIG. 32 illustrates tumor weight in nude mice transplanted human lung cancer cell lines (G2; A549) on the 21^{st} day of the experiment. Autopsy of the mice was performed on the 21^{st} day of the experiment.

FIG. 33 illustrates tumor weight in nude mice transplanted human colon cancer cell lines (G3; HCT15) on the 21^{st} day of the experiment. Autopsy of the mice was performed on the 21^{st} day of the experiment.

FIG. 34 illustrates tumor weight in nude mice transplanted human melanoma cancer cell lines (G4; LOX-IMVI) on the 21^{st} day of the experiment. Autopsy of the mice was performed on the 21^{st} day of the experiment.

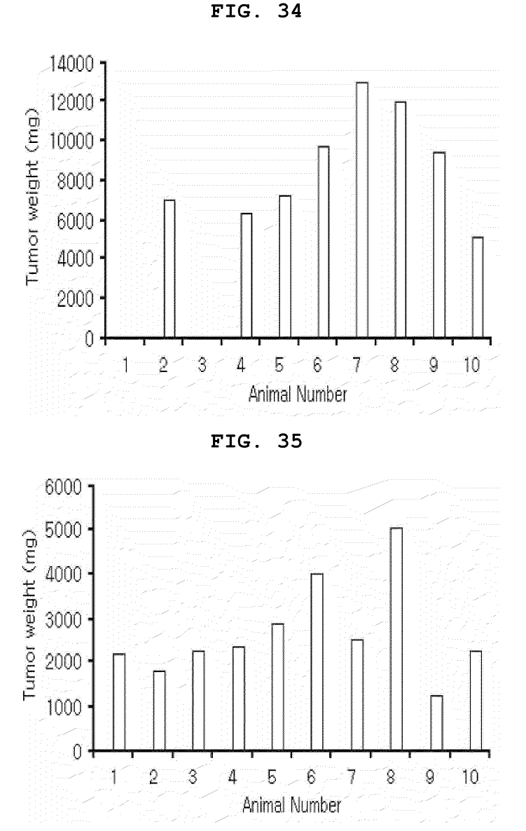

FIG. 35 illustrates tumor weight in nude mice transplanted human prostate cancer cell lines (G5; PC-3) on the 21^{st} day of the experiment. Autopsy of the mice was performed on the 21^{st} day of the experiment.

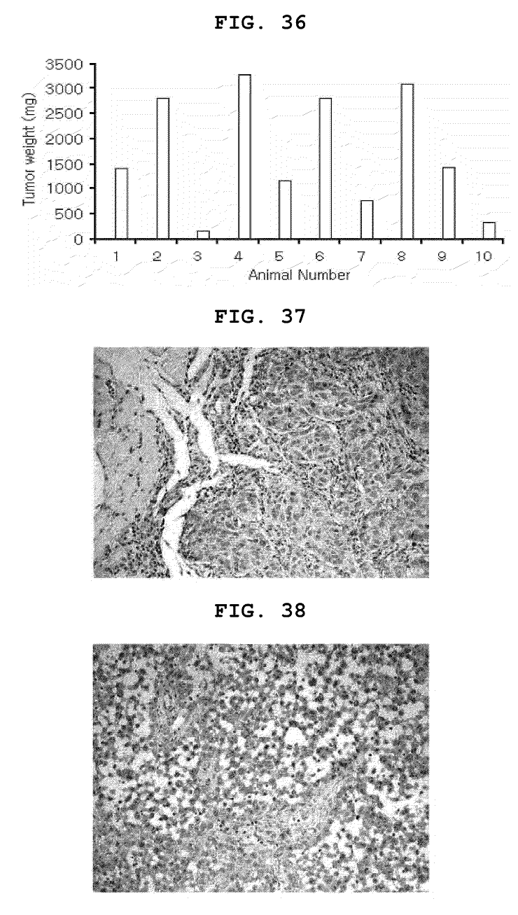

FIG. 36 illustrates tumor weight in nude mice transplanted human breast cancer cell lines (G6; MDA-MB-231) on the 21^{st} day of the experiment. Autopsy of the mice was performed on the 21^{st} day of the experiment.

FIGS. 37 through 41 illustrate histopathological findings in nude mice transplanted human cancer cell lines. FIG. 37 illustrates well differentiated carcinoma of the lung cancer.

FIG. 38 illustrates manifest mitotic and gangrenous carcinoma of the colon cancer.

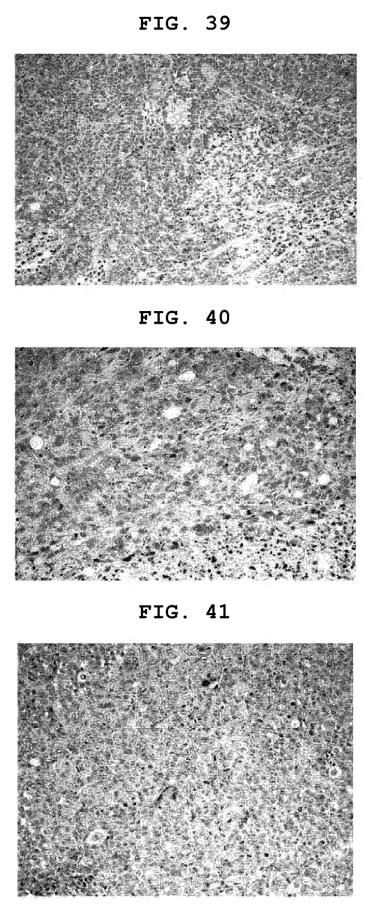

FIG. 39 illustrates gangrenous, undifferentiated and pleomorphic carcinoma of the melanoma.

FIG. 40 illustrates insufficiently differentiated but manifest gangrenous and pleomorphic carcinoma of the prostate cancer.

FIG. 41 illustrates insufficiently differentiated but sufficiently mitotic solid carcinoma of the breast cancer.

1. A real-time disease diagnostic system employing a non-invasive method by analyzing an electromagnetic field, and variations thereof, radiated from a subject, the system comprising: a sensor probe 1 comprising at least one biosensor-receiving a biological electro-magnetic field in a living organism including a human body and changing a capacitance; an analog circuit 2 for processing a bio-action potential signal measured by the sensor probe 1 into an analog signal; a digital conversion circuit 3 for converting the analog signal outputted from the analog circuit 2 into digital signal and processing the digital signal; and a power circuit 4 for supplying driving power to the system and charging a battery with power, a communication circuit for communicating with a PC, and a communication module 19 for wireless communication with a PC.

2. The system according to claim 1, wherein the analog circuit 2 comprises: a multi-channel multiplexer 22 for selecting a channel from the sensor probe 1; a sensor selection unit 23 for selecting specific sensors among multi-channel sensors from the sensor probe 1 and selecting a sensor for measurement from the specific sensors; a frequency controller 24 for minimizing errors such as an error caused when a region of the biosensors of the sensor probe 1 is composed of multiple channels, a basic manufacturing error of electronic components, and an environmental error of a measurement region and the like and adjusting a frequency generated in a frequency oscillation circuit 20 to a reference frequency of the sensor accurately; a frequency generator 25 for generating a reference frequency unique to the sensors based on the capacitance element when the biosensors in the sensor probe 1 is in a normal state before their diagnosis; a frequency signal amplifier 26 for amplifying level of a frequency signal generated from the frequency generator 25 up to a level where the frequency signal can be in the digital conversion circuit 3; and a frequency allocator 27 for allocating the frequency to be measured in the digital conversion circuit 3.

3. The system according to claim 1, wherein the digital conversion circuit 3 comprises:
a flash memory 12 for storing measurement data and program data;
a SDRAM 13 used for a temporary memory area;
a CPU 11 for measuring the frequency and performing various operations;
a PWM module 16 comprising a switch circuit receiving a command from a user and a buzzer 16b generating sounds according to an input frequency;
a LCD 15 for operating measured data and displaying the operated data on a graphic user interface (GUI); and
a LCD inverter 14 for controlling a brightness of the LCD 15.

4. The system according to claim 1, wherein the power circuit 4 is free from the use of power supplied from a commercial power source, the power is supplied from a battery 34 even though an adaptor 31 is used for connection, and
wherein the power circuit 4 is configured to feed back a voltage of the battery 34 to the CPU 11 by a battery charge measurement circuit 32 in order to check a residual capacity and charged capacity of the battery 34 and residual capacity of battery.

5. The system according to claim 2, wherein the frequency signal amplifier 26 converts a level of a frequency signal outputted through a RLC circuit in the frequency generator 25 up to a level where the frequency signal can be measured in the digital conversion circuit 3.

6. The system according to claim 1, wherein the basic capacitance of the biosensor is in a range from 0.5 pF to 900 pF.

7. The system according to claim 6, wherein a frequency of an oscillating circuit is determined according to the changes in the capacitance of the biosensor.

8. The system according to claim 1, wherein a value of the capacitance is converted into a frequency or a voltage value for diagnosing a disease of the subject, wherein the capacitance is changed according to a value of the bio-action potential detected and inputted by the biosensor from a living tissue of the subject.

9. The system according to claim 1, wherein the health condition of the subject is determined according to a variation of a delta frequency which is a difference value between the reference frequency unique to the biosensor and the measured frequency detected by the biosensor.

10. The system according to claim 9, wherein the difference value between the reference frequency and the measured frequency is determined by determining a delta frequency using a clock of the CPU 11 supplied through an oscillator 10 and a voltage-frequency (VF) converter, measuring the frequency oscillated from the sensor, and comparing the measured frequency with the reference frequency.

11. The system according to claim 9, wherein the difference value between the reference frequency and the measured frequency is determined by converting a voltage value into a digital value and comparing the measured frequency with the reference frequency, the voltage value being obtained by passing the frequency outputted from the sensor through a frequency-voltage (FV) converter using an A/D converter inside or outside of the CPU 11 in measuring the frequency oscillated from the sensor.

12. The system according to claim 9, wherein the reference frequency is a reference frequency for comparing with the measured frequency detected by the biosensor when the biosensor is free from the exposure to the bio-action potential, and which is adjusted to the same frequency as the frequency unique to the biosensor.

13. The system according to claim 12, wherein the reference frequency of the sensor as the frequency unique to the biosensor has a frequency band range from 0.5 Hz to 95 MHz.

14. The system according to claim 13, wherein the reference frequency is primarily calibrated in the analog circuit and secondarily adjusted to the same frequency as the frequency unique to the biosensor by the digital conversion circuit and a given program, in consideration of the characteristics of the biosensor sensitive to measurement environments.

15. The system according to claim 14, wherein the reference frequency that can be adjusted by an analog mode is in a range from 5 Khz to 10 Mhz.

16. The system according to claim 14, wherein the reference frequency that can be adjusted by a digital mode is in a range from 0.1 hz to 1 Mhz.

17. The system according to claim 5, wherein an acquisition speed of the value of bio-action potential inputted from the biosensor to be changed by the digital mode.

18. The system according to claim 9, wherein the health condition of the subject is classified into three types or more according to the measured value of the delta frequency, which are represented by green, yellow, red color, or other diverse color indicators, respectively.

19. The system according to claim 9, wherein the health condition of the subject is classified into three types or more according to the measured value of the delta frequency which is shown in color as green, yellow, red color, or other diverse color indicators, respectively, and, in the case of the subject suffering from cancer, the yellow and red indicators are irregularly displayed on an LCD screen and displayed on a screen of a monitor of the PC to which data measured by the measurement device is transmitted.

20. The system according to claim 16, wherein the health condition of the subject is classified into three types or more according to the measured value of the delta frequency which is shown in color as green, yellow, red color, or other diverse color indicators, respectively, and boundary values among green, yellow, red color and other various colors is controlled according to the purpose of use.

21. The system according to claim 9, wherein the health condition of the subject is classified into multiple types according to the measured value of the delta frequency which is shown in color as a sound signal or an alarm sound having the frequency different from one another.

22. The system according to claim 1, wherein the disease diagnostic system is connected with the PC by means of a wireless LAN, a wireless communication module, a USB port or RS-232C, and the PC program the data measured by the disease diagnostic system in the form of a three-dimensional graphic, multi-dimensional graphic or stereoscopic image.

23. The system according to claim 1, wherein the disease diagnostic system is connected with the PC by means of a wireless LAN, a wireless communication module, a USB port or RS 232C, and the PC stores the data measured by the disease diagnostic system into the database.

24. The system according to claim 17, wherein the health condition of the subject is classified into three types or more according to the measured value of the delta frequency which is shown in color as green, yellow, red color, or other diverse color indicators, respectively, and boundary values among green, yellow, red color and other various colors is controlled according to the purpose of use.
